(19) United States
(12) Patent Application Publication
(10) Pub. No.: US 2015/0219636 A1
(43) Pub. Date: Aug. 6, 2015

(54) ISOLATION OF CELLS AND BIOLOGICAL SUBSTANCES USING BUOYANT MICROBUBBLES

(71) Applicant: Targeson, Inc., San Diego, CA (US)

(72) Inventors: Joshua L. Rychak, Oceamside, CA (US); Dan J. Smith, San Diego, CA (US); Alice Luong, San Diego, CA (US); B. Jack DeFranco, La Jolla, CA (US)

(73) Assignee: Targeson, Inc., San Diego, CA (US)

(21) Appl. No.: 14/608,177

(22) Filed: Jan. 28, 2015

Related U.S. Application Data
(60) Provisional application No. 61/965,403, filed on Jan. 28, 2014.

(51) Int. Cl. G01N 33/543; B01L 3/00; G01N 33/569

(52) U.S. Cl. CPC G01N 33/5432; G01N 33/56972; B01L 3/5021; G01N 2333/70517; G01N 2333/70514; G01N 2333/70503; B01L 2200/0647; B01L 2300/0832; B01L 2300/087; B01L 2400/0409

(57) ABSTRACT

Methods, compositions and a two-chamber apparatus are provided for use in the separation of a biological substances type from a complex liquid mixture utilizing buoyant microbubble compositions.

### CROSS-REFERENCE TO RELATED APPLICATIONS

This application claims the benefit of priority to U.S. Provisional Application No. 61/965,403 filed on Jan. 28, 2014, the contents of which is incorporated herein by reference.

### BACKGROUND OF THE INVENTION

Methods for isolating a desired cell type from a complex mixture are useful in a number of biomedical fields. In basic and applied biomedical research, the need for cell purification and separation techniques is widespread and underpins a wide variety of types of experiments. One common application pertains to basic immunological research, where a scientist desiring to study a specific type of leukocyte must isolate the desired cell from whole blood. Another application pertains to a clinical application of gene therapy, wherein cells are harvested from a patient and treated to express the desired gene. The cells successfully expressing the gene are then purified from non-responsive cells, and administered back to the patient, thereby affecting the therapy. Yet another application involves isolating or quantifying cytokines found at low concentrations in body fluids, for example breast milk. Finally, in some applications depletion may be the end goal. For example, it may be desirable to remove dead cells during cell culture, or to remove a soluble factor from a population of cells.

For virtually all practical purposes, a separation technique must be rapid (completed in minutes), innocuous to the cells, and result in a usable yield, purity, and viability of isolated cells. In the case in which removal of undesired cells form the starting material is intended, depletion efficiency is a key performance metric. Values for usable purity and yield can be obtained from products currently in commercial use: yield of ˜50% and purity of 90-99% are generally considered sufficient. Viability of >90% is generally sufficient. In the case of depletion, >90% depletion efficiency is generally considered sufficient.

TABLE 1
Representative products used for cell separation.

| | | | Manufac- | | |
|---|---|---|---|---|---|
| Target Cell | Tissue | Product | turer | Cat # | Purity |
| CD4+ | Human | EasySep | StemCell | 18052 | 98.8% |
| lymphocytes | PBMC | human | Technol- | | |
| (helper | | CD4+ | ogies | | |
| T-cells) | | Isolation Kit | | | |
| CD8+ | Human | EasySep | StemCell | 18053 | 99.6% |
| lymphocytes | PBMC | human | Technol- | | |
| (cytotoxic - | | CD8+ | ogies | | |
| cells) | | Isolation Kit | | | |
| Monocytes | Human | FlowComp | Life | 11367D | 99% |
| | PBMC | Human | Technol- | | |
| | | CD14 | ogies | | |
| CD19+ | Human | EasySep | StemCell | 18054 | 98.5% |
| lymphocytes | PBMC | human | Technol- | | |
| (B-cells) | | CD19+ | ogies | | |
| | | Isolation Kit | | | |
| CD56+ | Human | EasySep | StemCell | 18055 | 98.1% |
| lymphocytes | PBMC | human | Technol- | | |
| (Dendritic | | CD56+ | ogies | | |
| cells) | | Isolation Kit | | | |
| CD34+ | Human | EasySep | StemCell | 18056 | 96.0% |
| (hemato- | cord | human | Technol- | | |
| poietic | blood | CD34+ | ogies | | |
| stem cells) | | Isolation Kit | | | |
| CD90.2+ | Mouse | Flow Comp | Life | 11465D | 97% |
| (lymphocyte) | spleno- | Mouse Pan-T | Technol- | | |
| | cytes | Kit | ogies | | |
| CD4+ | Mouse | EasySep | StemCell | 19852 | 96.7% |
| lymphocytes | spleno- | Mouse | Technol- | | |
| (helper | cytes | CD4+ | ogies | | |
| T-cells) | | Isolation Kit | | | |
| CD8+ | Mouse | EasySep | StemCell | 19853 | 91.5% |
| lymphocytes | spleno- | mouse CD8+ | Technol- | | |
| (cytotoxic - | cytes | Isolation Kit | ogies | | |
| cells) | | | | | |
| Monocytes | Mouse | EasySep | StemCell | 19761 | 88% |
| | bone | mouse | Technol- | | |
| | marrow | Monocyte | ogies | | |
| | | Enrichment | | | |
| | | Kit | | | |
| CD 19+ | Mouse | EasySep | StemCell | 19854 | 97.7% |
| lymphocytes | spleno- | mouse | Technol- | | |
| (B-cells) | cytes | B-Cell | ogies | | |
| | | Isolation Kit | | | |

Several separation reagents have been developed for cell separation and purification over the last several decades. These can be broadly classified into receptor-based, wherein the expression of specific surface molecules is used to identify desired from undesired cells, and gradient-based, wherein differences in the movement of cell types through a liquid medium with variable density and/or viscosity is used to isolate the desired cell type. Both types of separation methods are now well-established, with numerous commercial incarnations currently in the market. In general, however, existing methods suffer from both difficulty or high cost in performance, and the potential for causing unwanted changes to the desired cells being harvested. An ideal cell separation technique would be easy and rapid to perform and would not cause changes to the desired cells.

Density gradient separation is commonly used for isolating desired cells from blood. This technique relies upon differential movement of cells through one or more layers of liquid media, such as Ficoll or Percoll, each having a slightly different density. This procedure is usually performed in a column, and consists of carefully placing the cell mixture upon the meniscus of media, followed by centrifugation for 10-45 min to speed the movement of cells through the column. Cells migrate through the media based upon their inherent density, forming layers within the column. The desired cells are isolated by collecting the desired layer from the column. This technique suffers from both the requirement for a high degree of technical skill, and also a relatively long performance time.

The specific expression of cell surface markers can be exploited for the purpose of selective isolation of desired cells. Antibodies or other molecules able to specifically bind a desired marker can be used to label desired cells for subsequent isolation. For example, fluorescence-activated cell sorting (FACS) uses antibodies bearing a fluorophore, which labels the desired cells fluorescently. A flow cytometer can then be used to isolate the desired cells based upon the increased fluorescence of the labeled cells. This is generally a time consuming procedure requiring up to hours for a single experiment, and requires access to expensive equipment.

Antibodies may also be conjugated to magnetic microparticles or nanoparticles. Upon mixing with a heterogeneous cell mixture, the magnetic particles are bound to the targeted cell. Magnetically labeled cells or solutes may then be isolated by passage through a magnetic field. This concept is implemented in several commercial products, including Dynabeads, and MACS®. Drawbacks pertaining to magnetic separation are damage to cells caused by the microbeads or columns and changes in cell phenotype caused by exposure to the beads (for example phagocytosis of the beads; Moore et al, 1997; Faraji et al, 2009; Pisanic et al, 2007; Berry et al, 2003) and the difficulty in removing the beads from the targeted cells. To date, however, magnetic isolation methods are widely used in many research and some clinical applications.

### BRIEF SUMMARY OF THE INVENTION

The present invention provides a method for isolating cells and other biological substances of interest using a novel buoyancy-based method. The advantages of the buoyancy-based method include inherent scale-ability (particularly with respect to magnetic methods), ease of implementation into existing research work flows, the ability to perform the method in a closed-loop system, the ease with which the separation reagent can be removed, and the ability to do multi-step positive selection isolations.

In the present invention, gas encapsulated microbubbles are contemplated as a reagent for buoyancy-based isolation. Such microbubbles can be prepared of lipids, proteins, and other generally biocompatible materials and coated with ligands specific for materials of interest. Such microbubbles have been broadly described in the context of medical imaging contrast agents. However, microbubbles that are suitable for medical imaging generally are not suitable for use as separation reagents due to the potential for the shell materials to activate isolated cells, the propensity for inducing aggregation, and the inability to fully remove the microbubble components from the targeted cells. Moreover, buoyant microbubbles bound to biological materials (including cell) tend to form a thin floating layer at the air-liquid interface following buoyancy-based separation, and complete collection of this floating layer is technically difficult.

In some embodiments, the present invention provides for a microbubble composition that can be used as a buoyancy-based separation reagent without the unwanted effects of previously described microbubble compositions, including cell aggregation, cell activation, or the presence of residual microbubble components on the cell.

In some embodiments, the present invention provides for a method of using buoyant microbubbles for buoyancy-based isolation using a two-chamber device. This method overcomes the inherent difficulty in collecting the buoyant and sedimented cell fractions.

In some embodiments, the present invention provides for a method of isolating biological substances suitable for use in diagnostic or therapeutic settings in a sterile closed system.

In some embodiments, the present invention provides for isolation of cells by positive selection without causing unwanted perturbation to the cells.

In some embodiments, the present invention provides for a method of isolating cells or soluble analytes comprising a procedure duration of 15 minutes or less.

In one aspect of the invention, efficacious collapse and removal of the microbubble is enabled by use of gas core contents that have moderate to high solubility in water.

In another aspect of the invention, efficacious collapse of the microbubble is enabled by the use of shell components that confer a phase transition temperature of between 10-35 degrees Celsius to the shell.

In another aspect of the invention, efficacious collapse of the microbubble is enabled by the use of shell components designed to have high water solubility. In another aspect of the invention, removal of the microbubbles from the attached cells is achieved by utilization of anchor compounds that can be readily removed from the microbubble shell upon collapse. In another aspect of the invention, robust performance as a separation reagent is conferred by maintaining a ligand density of between 1-50,000 molecules per MB.

In another aspect of the invention, robust performance as a separation reagent is conferred by selecting shell components that do not adversely perturb cells in a single cell suspension, nor interfere with commonly used downstream assays.

In another aspect of the invention, robust performance as a separation reagent is conferred by selecting shell forming materials that have no net charge.

### BRIEF DESCRIPTION OF THE DRAWINGS

FIG. 1: Schematic (side view) example of the two-chamber device, in the orientation for use in loading the Test Sample into the bottom chamber. Arrow in upper right corner indicates the direction of gravity. Panel i) illustrates the apparatus disassembled, with key components labeled: (A) Lid for upper chamber, (B) upper chamber, into which the buoyant sample will be transferred during the separation process. The upper chamber is loaded with collection buffer. (C) Insert, attached to the interior wall of the apparatus, comprising (D) a narrow opening and (E) a wide opening (F) Extender, that connects the wide opening of the insert to, (G) Bottom chamber, in which the non-buoyant sample will become concentrated during the separation process. The Test Sample is loaded into the bottom chamber. Panel ii) illustrates the apparatus assembled, with key components labeled: (H) Upper chamber lid is attached to the upper chamber, forming an airtight seal. (I) Dotted line represents the air-liquid interface, with said liquid comprising the collection buffer. (J) Bottom chamber is attached to the Extender, forming an airtight seal. (K) Bottom of the bottom chamber is tapered. Panel iii) illustrates the apparatus assembled, with key distances labeled: (L) Distance between the air-liquid interface (I) and the upper chamber lid (A), (M) Distance between the narrow end of the insert (D) and the air-liquid interface (I), (N) Distance between the narrow end of the insert (D) and the bottom of the bottom chamber (K), (O) Distance between the bottom of the bottom chamber (K) and the seal between the extender and bottom chamber (J), (P) Distance between the wide end of the insert (E) and the seal between the extender and bottom chamber (J).

FIG. 2: Schematic example of the two-chamber device, in the orientation for use in loading the Test Sample into the upper chamber. Panel i) illustrates the apparatus disassembled, with key components labeled: (A) Lid for upper chamber, (B) Upper chamber, into which the buoyant sample will be transferred during the separation process. The Test Sample is loaded into the upper chamber. (C) Insert, attached to the interior wall of the apparatus, comprising (D) a narrow opening and (E) a wide opening. (F) Extender, that connects the wide opening of the insert to, (G) Bottom chamber, in which the non-buoyant sample will become concentrated during the separation process. Collection buffer is loaded into the bottom chamber. Panel ii) illustrates the apparatus assembled, with key components labeled: (H) Upper chamber lid is attached to the upper chamber, forming an airtight seal. (I) Dotted line represents the air-liquid interface, with said liquid comprising the Test Sample. (J) Bottom chamber is attached to the Extender, forming an airtight seal. (K) Bottom of the bottom chamber is tapered. Panel iii) illustrates the apparatus assembled, with key distances labeled: (L) Distance between the air-liquid interface (I) and the upper chamber lid (A), (M) Distance between the narrow end of the insert (D) and the air-liquid interface (I), (N) Distance between the narrow end of the insert (D) and the bottom of the bottom chamber (K), (O) Distance between the bottom of the bottom chamber (K) and the seal between the extender and bottom chamber (J), (P) Distance between the narrow end of the insert (E) and the seal between the extender and bottom chamber (J).

FIG. 3: Schematic of the assembled two-chamber device, with key measurements indicated. (A) Diameter of the upper chamber at the widest point, (B) Diameter of the insert at the wide end, (C) Diameter of the insert at the narrow end, (D) Diameter of the extender at the widest point, (E) Diameter of the bottom chamber at the most narrow point.

FIG. 4: Schematic depicting loading of the bottom chamber first, for apparatus in which the test sample is loaded into the upper chamber. The upper chamber is first detached from the lower chamber. (A) Loading of the bottom chamber with collection buffer. The top chamber is then loaded (B) with the test sample. A slight amount of collection buffer (D) may be present in the upper chamber. The top lid is then attached to the upper chamber with an airtight seal (E). (F) The air-liquid interface in the upper chamber.

FIG. 5: Schematic depicting loading of the bottom chamber first, for apparatus in which the test sample is loaded into the bottom chamber. The upper chamber is first detached from the lower chamber. (A) Loading of the bottom chamber with the test sample. The upper chamber is then attached to the lower chamber forming an airtight seal (C). The top chamber is then loaded (B) with collection buffer. A slight amount of test sample (D) may be present below the narrow end of the insert. The top lid (E) is then attached to the upper chamber with an airtight seal. (F) The air-liquid interface in the upper chamber.

FIG. 6: Schematic depicting loading of the top chamber first, for apparatus in which the test sample is loaded into the bottom chamber. The bottom chamber is attached to the top chamber, and the lid of the top chamber is removed. (A) Loading of the top chamber (B) with collection buffer. The top lid (E) is then secured to the top chamber with an airtight seal. The apparatus is then inverted, and the bottom chamber (D) removed. (F) The test sample is loaded into the lower chamber. The bottom lid (D) is then secured with an airtight seal, and the apparatus inverted again. The isolation procedure is now ready for the separation procedure.

FIG. 7: Schematic depicting loading of the bottom chamber first, for apparatus in which the test sample is loaded into the top chamber, and in which detachment of the bottom chamber is not required. (A) Collection buffer is added to the bottom chamber (C), through the insert. (D) The test sample is loaded into the upper chamber. A small amount of collection buffer may extend into the upper chamber (E). (G) Shows the air-liquid interface in the upper chamber.

FIG. 8: Schematic depicting loading of the bottom chamber first, for apparatus in which the test sample is loaded into the bottom chamber, and in which detachment of the bottom chamber is not performed. (A) The test sample is added to the bottom chamber (C), through the insert. (D) Collection buffer is loaded into the upper chamber. A small amount test sample may extend into the upper chamber (E). (G) Shows the air-liquid interface in the upper chamber.

FIG. 9: Schematic depicting direction of movement of the buoyant fraction in an apparatus in which the test sample is loaded into the bottom chamber (B). During the separation procedure, the buoyant materials are transferred from the bottom chamber (B), through the insert, and into the top chamber (A). Arrow C shows the direction of buoyant material motion (microbubbles and microbubble-cell complexes).

FIG. 10: Schematic depicting direction of movement of the non-buoyant fraction in an apparatus in which the test sample is loaded into the top chamber (A). During the separation procedure, the non-buoyant materials are transferred from the top chamber (A), through the insert, and into the top chamber (B).

FIG. 11: Schematic depicting method for collapse of microbubbles in the two chamber apparatus. A pressure-generating device (C) is connected (B) to the upper chamber via a port (A).

FIG. 12: Schematic (oblique view) example of the two-chamber device, in the orientation for use in loading the Test Sample into the bottom chamber. Arrow in upper right corner indicates the direction of gravity. Panel i) illustrates the apparatus disassembled, with key components labeled: (A) Lid for upper chamber, (B) Upper chamber, into which the buoyant sample will be transferred during the separation process. The upper chamber is loaded with collection buffer. (C) Insert, attached to the interior wall of the apparatus, comprising (D) a narrow opening and (E) a wide opening, (F) Extender, that connects the wide opening of the insert to, (G) Bottom chamber, in which the non-buoyant sample will become concentrated during the separation process. The Test Sample is loaded into the bottom chamber.

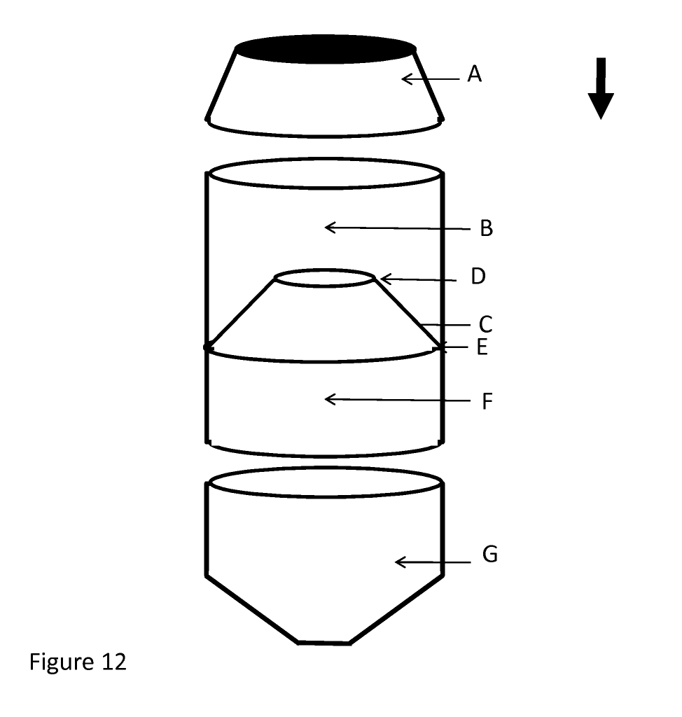

FIG. 13: Plot depicting the relationship between anchor density and ligand density for two representative targeting ligands. Microbubbles were prepared following the method of Example 3, using an anchor density of between 0.01% and 1.0% (by moles). An anti-PE antibody (Ligand 1) and an anti-APC monoclonal antibody (Ligand 2) were conjugated to the microbubble surface following the method of Example 3. Ligand density was determined by ELISA, using known concentrations of each ligand as a concentration standard.

FIG. 14: Demonstration of absence of unwanted cellular perturbation in response to microbubbles synthesized in accordance with the instant invention. Microbubbles comprising an anti-CD11b antibody were synthesized as in Examples 2-3. Microbubbles were incubated with a mouse monocytic cell line (RAW264.7) and various assays designed to assess functional alterations in the cells were performed. (a) Cells were incubated with increasing concentrations of MB (between 0.1 and 100 per cell), or ethanol (EtOH) as a positive control. Cell viability was assessed at 2 hours by 7AAD, (b) Cells were incubated with buffer alone or MB (10 MB per cell) and proliferation was assessed at 24 hours, (c) Cells were incubated with buffer alone, MB without a targeting ligand (10 MB per cell), or MB bearing the CD11b antibody (10 MB per cell). Release of NO was assessed before or after stimulation with LPS. This panel of assays demonstrated no functional alterations in the cells following contact with the microbubbles. Error bars represent standard deviation for at least n=3 replicates

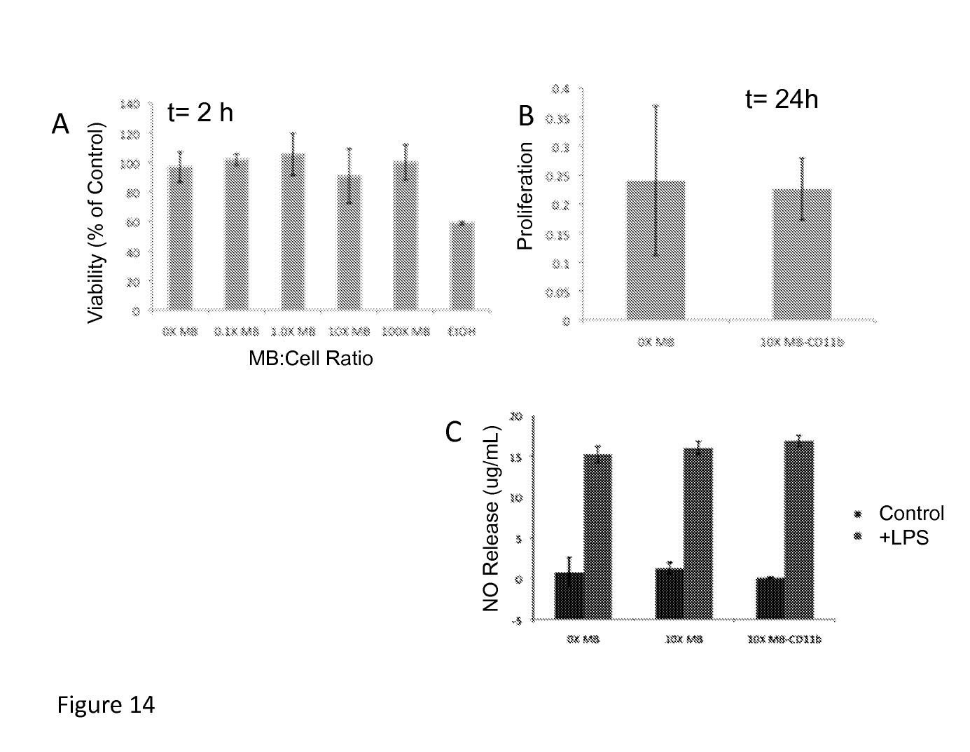

FIG. 15: Demonstration of irreversible cell-cell aggregation due to targeted MB. Fresh mouse splenocytes were stained with a PE-labeled anti-CD19 antibody. Cells were then incubated with a microbubble bearing an anti-PE antibody (10 MB per cell), prepared to have a high antibody density (˜50,000 molecules per MB) for 5 minutes. Cells were then isolated by positive selection and microbubbles collapsed by positive selection. The positive fraction was observed on a hemacytometer before microbubble collapse (A) and after collapse (B). Significant cell-cell aggregates were observed both before and after collapse relative to untouched cells that had not been incubated with MB (C). (D) Quantification by cell counting after MB collapse revealed that a significant proportion of the cells that were in contact with MB (up to 40%) were in irreversible aggregates that persisted after microbubble collapse.

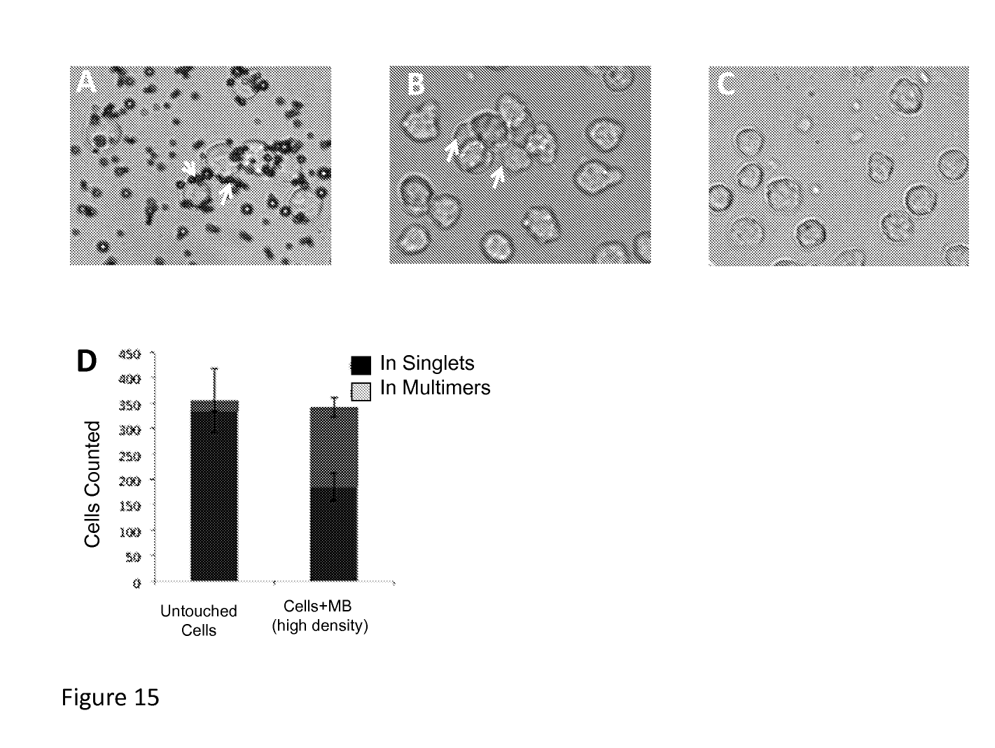

FIG. 16: Modulation of cell aggregation by MB:Cell incubation ratio. Fresh mouse splenocytes were stained with an PE-conjugated anti-CD19 antibody, and subsequently incubated with microbubbles comprising an anti-PE antibody at a MB:Cell ratio of 0 (buffer alone), 1, or 10. The number of cells in aggregates, and the number of cells within each aggregate, was significantly greater for upon incubation with 1× relative to 10×MB.

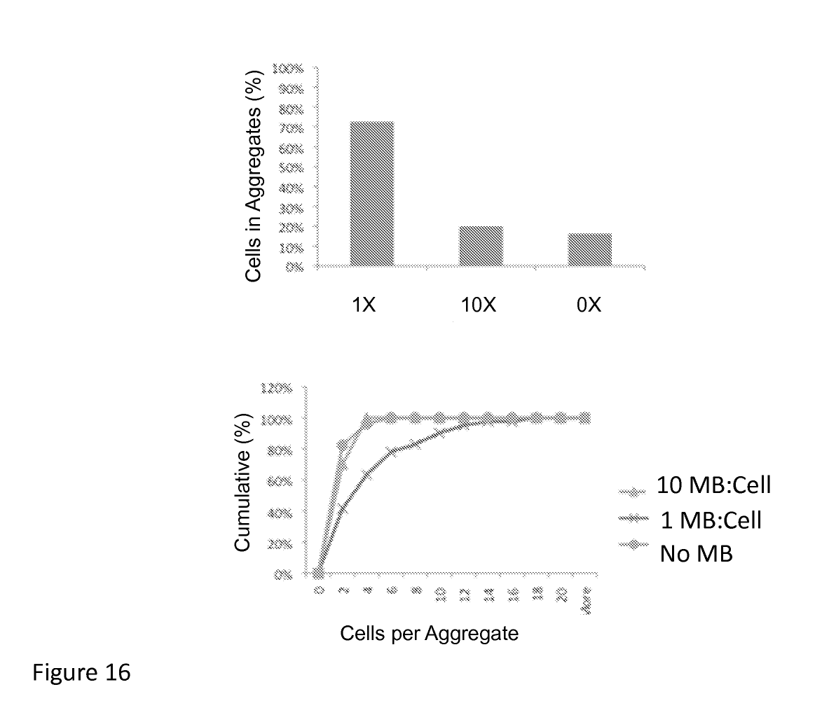

FIG. 17: Modulation of technical loss by ligand density and MB:Cell incubation ratio. Fresh mouse splenocytes were stained with APC-conjugated anti-CD19 antibody, and subsequently incubated with microbubbles comprising an anti-APC antibody. MB were synthesized with various densities of antibody, as indicated on the x-axis. Each MB formulation was incubated with cells at a MB:Cell ratio of 1, 10, or 40. Technical loss was determined by counting flow cytometry. The data demonstrate that technical loss increases with antibody density. Moreover, for a given antibody density the technical loss can be reduced by adding a higher ratio of MB to cells. Corresponding microscopy revealed that technical loss in this experiment was primarily due to formation of irreversible aggregates in the positive cell fraction.

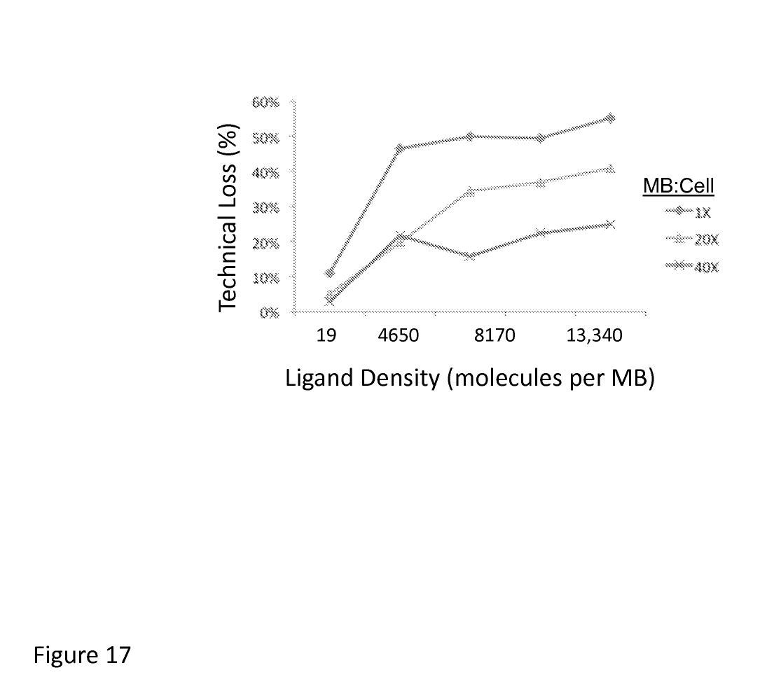

FIG. 18: Improvement in cell separation performance using MB with lower antibody density. MB comprising an anti-APC antibody conjugated at an anchor density of 0.25% or 0.1% were synthesized. Positive selection of fresh mouse splenocytes stained with an APC-conjugated anti-CD19 antibody was performed in triplicate, and key performance parameters were computed. It was observed that depletion and yield were significantly higher for MB comprising the lower anchor density, and technical loss was significantly lower. Error bars represent standard deviation of 3 experiments.

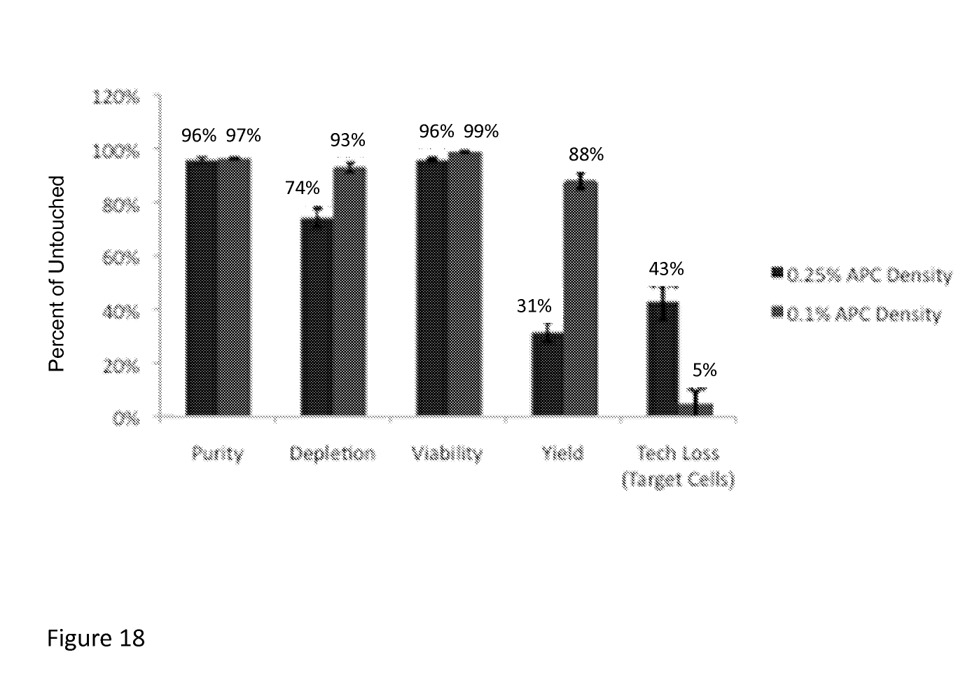

FIG. 19: Demonstration of improved collapsibility for microbubbles above the phase transition temperature. Microbubbles comprising a fluorophore inserted into the shell for the purpose of visualizing the shell with high resolution were prepared, and imaged by epifluorescence microscopy. A identical concentration of MB was used for each experiment. Microbubbles had an initial mean diameter of ˜2.5 um. A) Intact MB appeared spherical by microscopy. B) Upon collapse by positive hydrostatic pressure, and at a temperature below the main lipid phase transition temperature (in this case, 8 deg C), the collapsed microbubble shell was observed to take the form of micron-scale strings, tubes, and larger aggregates. C) Upon collapse by positive hydrostatic pressure, and at a temperature above the main lipid phase transition temperature, the collapsed MB shell adopted primarily sub-visible and undetectable structures, with occasional small vesicles.

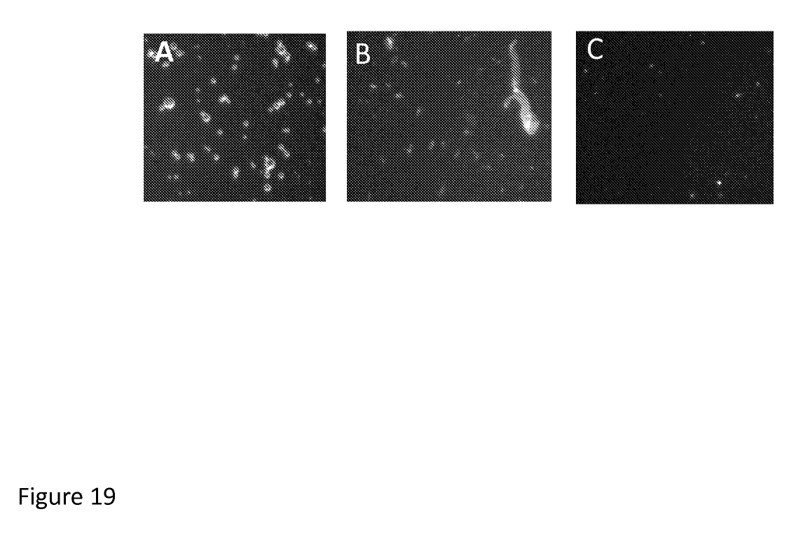

FIG. 20: Demonstration of high-purity separation of buoyant microbubbles from free cells with the use of a two-chamber device. The relative concentration of cells and microbubbles was determined by flow cytometry. Fresh mouse splenocytes were incubated with naked (no targeting ligand) microbubbles, then placed into the upper chamber of a two-chamber device and centrifuged. The contents of the upper and lower chambers were assessed by flow cytometry, and the concentration of microbubbles and cells in each chamber was quantified. This experiment was repeated 3 times (n=3). (A) Forward-side scatter plot derived from flow cytometry reveals the presence of both cells (rectangle gate) and microbubbles (oval gate) in the upper chamber before centrifugation. After centrifugation and separation of the two chambers, the upper chamber (B) is shown to be highly enriched in microbubbles, while (C) the lower chamber is enriched in cells. Quantification of the concentration of cells and microbubbles in each chamber shows essentially all microbubbles were retained in the upper chamber and essentially all cells were transferred to the bottom chamber.

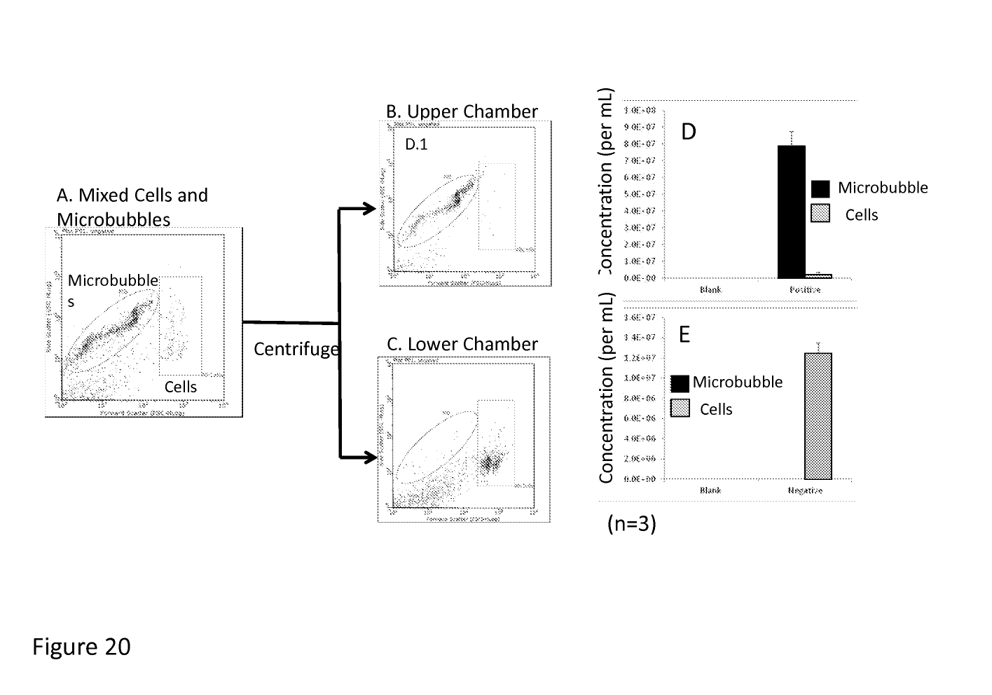

FIG. 21: Example of using a streptavidin-bearing microbubble for isolation of CD4+ cells from a complex mixture consisting of spleen homogenate. Splenocytes were incubated with a biotinylated CD4 antibody, unbound antibody removed by washing the cells, then the cells were incubated with a streptavidin-coated microbubble. After centrifugation, the upper and lower fractions were assessed for the presence of the targeted CD4 cells by flow cytometry. This experiment was repeated n=4 times. (A) CD4 cells comprise approximately 15% of the mixed cell population. (B) After applying our separation method, CD4 cells are enriched to >90% purity. (C) Quantification of the presence of CD4 cells in the positive and negative fractions show reproducibly high purity.

### DETAILED DESCRIPTION OF THE INVENTION

### I. Introduction

Several separation methods based on buoyant particles have been demonstrated. Buoyant microspheres composed of plastic (Delaage et al, U.S. Pat. No. 5,116,724) or hollow glass spheres (Hsu et al, 2010 and U.S. Pat. No. 8,513,032) conjugated to targeting ligands have been proposed. Removal of these particles from cells poses a difficulty in use of this technology, and the use of incompliant and rigid particles poses the potential for damage to cells.

Gas-encapsulated microbubbles, originally developed as contrast agents for ultrasound imaging, have also been investigated for buoyancy-based separation. Such microbubbles are generally pliant, biocompatible, and can be rendered non-buoyant by collapse.

Jablonski (U.S. Pat. Nos. 8,513,032 and 8,835,186) discloses microbubbles composed of denatured human albumin for isolation of bacteria and other biological materials by buoyancy. The microbubbles are coated with antibody or other affinity molecules that are intended to bind to the desired target cell or analyte. Drawbacks to this approach include the possibility for unwanted effects when using these reagents for non-human cells, and the high potential for non-specific binding of non-target cells to the denatured albumin shell. The use of lipid microbubbles generally overcomes these limitations, in that lipids are generally biocompatible across species and can be readily derivatized with polymers (such as polyethylene glycol) that can reduce non-specific binding to biological substances.

Klaveness (U.S. Pat. No. 6,261,537) notes that targeted microbubbles may be useful “diagnosis of different diseases or characterisation of different components in blood or tissue samples,” by binding cells to the microbubbles and effecting separation by flotation and repeated washing. Klibanov (U.S. Publication No. US20050260189) teaches similar targeted microbubbles for isolation of targeted cells by centrifuging in a syringe or flask. Cuthbertson (U.S. Publication No. 20030104359) and Rongved (U.S. Publication No. 20070036722) both disclose gas-encapsulated lipid microbubbles bearing targeting ligands for buoyancy-based isolation of cells. Wilson similarly (U.S. Publication No. 20120288852) discusses the use of targeted microbubbles for isolation of cells. Finally, microbubbles and methods for cell separation are taught by Kulseth (U.S. Pat. No. 6,806,045), Mattrey (International (PCT) Publication No. WO2009052057), and Simberg (2009).

Key performance parameters for cell separation are cell viability, purity, depletion, yield. The time required to perform the separation procedure may be important in some applications, with preference for more rapid performance. Disclosed methods have generally resulted in low yield, low purity, unduly long procedure time, or technical difficulty rendering routine use impossible. To date, no commercial products utilizing microbubble-based separation have emerged.

U.S. Patent publication Nos. US2007/0036722 and US2003/0104359 each disclose ligand-conjugated microbubbles for isolation of cells. However, the method disclosed in these publications results in low separation efficacy. Each reported a purity of between 50% (Cuthbertson, 0160) and 87% (Cuthbertson, 0110). This degree of enrichment is insufficient for practical use, and existing commercial products provide purities of >95% for these applications. Similarly, Simberg and Mattrey (2009) evaluated buoyant microbubbles for cell separation, and found that collection of the microbubble-bound cells was technically difficult, rendering positive selection unworkable in their system. Jablonski (U.S. Pat. No. 8,513,032) demonstrated capture of bacteria onto the surface of targeted microbubbles, but did not teach that targeted cells (bacteria) could be selectively captured from a mixed cell population (such as blood or tissue homogenate).

In each of the aforementioned works, the authors teach that microbubbles suitable for this application are comprised of formulations suitable for use as ultrasound contrast agents. That is, the microbubbles are composed of shell and gas components generally suitable for use in microbubble imaging agent formulations, and targeting ligands are conjugated to the microbubble surface following methods used in the same. The prior art in the field does not teach methods for practicing buoyancy-based separation suitable for practical use. Moreover, we discovered several specific characteristics that, when implemented into the microbubble formulation, render said microbubbles especially suitable for buoyancy-based separation. Surprisingly, these characteristics are particularly undesirable for microbubbles used as imaging agents, and in many cases render said microbubbles unusable for in vivo or imaging applications.

Prior art teaches that cells bound to microbubbles move to the top of the separation chamber, forming a buoyant layer distinct from the bulk fluid. It will be apparent to one skilled in the art that the concentrated microbubbles exhibit unique rheological properties that renders collection of the buoyant layer difficult. For example, International (PCT) Publication No. WO2009052057 states that “the bubbles with the attached cells form a foamy layer.” Simberg (2009) states that “it is technically challenging to collect all the MBs after separation,” and in this study only the negative (sedimented) fraction was able to be collected and analyzed. As discussed below, several solutions to this difficulty have been proposed, although none are suitable for achieving commercially-relevant performance in the setting of routine cell separation applications. The instant invention discloses a simple two-chamber apparatus for use in conjunction with microbubble-based cell separation; this apparatus enables buoyancy-based separations to be performed efficiently, rapidly, and with a high degree of robustness.

Prior art teaches that microbubbles used as imaging contrast agents are generally suitable for buoyancy-based separation applications. For example, Kulseth (U.S. Pat. No. 6,806,045) states that microbubbles “which are suitable for use in targetable contrast agent formulations, especially targetable ultrasound contrast agent formulations,” are useful for cell separations. As discussed below, microbubble formulations that are suitable as ultrasound contrast agents have physical properties that render then unsuitable for buoyancy-based separation applications. These properties are related to the composition of the microbubble, in particular the selection of shell material, gas or gas mixture, and type and density of targeting ligand. The instant application discloses microbubble compositions that are suited for buoyancy-based cell separation applications.

### II. Definitions

The abbreviations used herein have their conventional meaning within the chemical and biological arts.

As used herein, the term “separation reagents” refers to a class of products designed for isolating a targeted cell or soluble analyte from a complex mixture or mixed cell population. Separation Reagents include, collectively, density gradient columns, magnetic beads, paramagnetic beads, bead-based immunoassays, filters, and fluorescently-labeled targeting ligands for use with fluorescently activated cell sorting (FACS).

As used herein, the term “Mixed Cell Population,” or “Mixed Sample” or “Heterogeneous Mixture,” or “Complex Mixture” refers to a collection of cells or soluble analytes dispersed in a liquid or semi-solid medium, wherein said cells comprise different phenotypes or genotypes.

As used herein, the term “Test Sample” refers to the mixed sample after incubation with the buoyant microbubbles of the instant invention, but prior to the separation procedure.

As used herein, the term “Targeting Ligand” or “ligand” refers to any material or substance that may promote targeting of tissues, cells, receptors, and/or marker groups in vitro or in vivo with the compositions of the present invention. The terms “target(s)”, “targeted” and “targeting”, as used herein, refer to the ability of targeting ligands and compositions containing them to bind with or be directed towards tissues, cells and/or receptors. The targeting ligand may be synthetic, semi-synthetic, or naturally-occurring. Materials or substances which may serve as targeting ligands include, for example, proteins, including antibodies, glycoproteins and lectins, peptides, polypeptides, saccharides, including mono- and polysaccharides, vitamins, steroids, steroid analogs, hormones, cofactors, bioactive agents, and genetic material, including nucleosides, nucleotides and polynucleotides.

As used herein, the term “Cell Surface Target”, or “Cell Surface Receptor”, refers to a structure on the surface of the cell that is characterized by the selective binding of a specific substance. Exemplary cell surface targets include, for example, cell-surface receptors for peptide hormones, neurotransmitters, antigens, complement fragments, immunoglobulins, cytoplasmic receptors for steroid hormones, cluster of differentiation designated molecules such as CD8, CD127, CD25, CD34, CD14, CD68, CD19, CD20, CD11b, CD11c, GR1, CD3, CD56, CD209, CD45, 71, CD61, CD41, CD31, CD133, surface markers of apoptosis such as phosphatidylserine, and cell adhesion molecules such as integrins.

As used herein, the term Collection Buffer refers to a the buffer solution into which the separated substances will be placed.

As used herein, the term “Active Substance” refers to a compound that is administered to a cell for the purpose of changing the phenotype or genotype of said cell. Exemplary active substances are plasmid DNA, siRNA, small molecule drugs, antibodies, and other therapeutic compounds.

As used herein, the term “Cell-Microbubble Complex” is defined as a complex consisting of one or more cells bound to one or more microbubbles, wherein said binding occurs via a targeting ligand attached to the microbubble and the cell surface target on the cell.

As used herein, the term “Soluble Analyte” is defined as a substance of interest dispersed in a liquid medium. Exemplary soluble analytes are nucleic acids, lipids, sugars, hormones, antibodies, cytokines, virus, virions, organelles, and other excreted biological substances.

As used herein, the term “Therapeutic Substance” refers to any therapeutic or prophylactic agent that may be used in the treatment (including the prevention, diagnosis, alleviation, or cure) of a malady, affliction, disease or injury in a patient or animal model. Therapeutically useful peptides, polypeptides and polynucleotides may be included within the meaning of the term therapeutic substance.

As used herein, the term “Positive Selection” refers to the application of a cell separation procedure in which the desired cells are found in the Positive Fraction.

As used herein, the term “Negative Selection” refers to the application of a cell separation procedure in which the desired cells are found in the Negative Fraction.

As used herein, the term “Isolation” refers to the act of isolating or completely separating one or more populations of cells from another population, and can refer to either Positive or Negative Selection.

As used herein, the term “Depletion” refers to the removal of an undesired substance from a biological sample, and in which the buoyant fraction is discarded and the non-buoyant fraction comprises the desired final product.

As used herein, the term “Purity” refers to the number of desired cells in the collected fraction relative to all of the cells in the collected fraction.

As used herein, the term “Yield” refers to the number of desired cells in the collected fraction relative to the number of desired cells in the mixed cell population before the cell separation procedure.

As used herein, the term “Viability” refers to the number of cells in the collected fraction that are viable relative to the total number of cells in the collected fraction. Viability may be measured using any standard metrics, including Trypan Blue exclusion, staining with 7AAD, and cell scattering properties on flow cytometry.

As used herein, the term “Depletion” refers to the number of targeted cells in the negative fraction relative to the number of targeted cells in the mixed cell population before the cell separation procedure.

As used herein, the term “Technical Loss” refers to the number of desired cells collected in the positive and negative fractions relative to the number of desired cells in the mixed cell population before the separation procedure.

As used herein, the term “Targeted Cell” refers to the cell type to which the targeting ligand on the microbubble is intended to bind. The relationship between the Targeted Cell and Cell Surface Target is defined as follows: the cell surface target is found on the target cell. In some applications, multiple Targeted Cells may be used.

As used herein, the terms “Non-Targeted Cell” or “Sedimented Cell” or “Non-buoyant Cell” refer to the cell type or types upon which the Cell Surface Target is not found, and to which the microbubbles do not bind, and which are not enriched in the buoyant fraction.

As used herein, the term “Two Chamber Device” refers to a container into which the microbubbles and liquid sample can be placed, which can be sealed to prevent movement of air or liquid, and which can be separated into an Upper Chamber and a Lower Chamber.

As used herein, the terms “Lower Portion” or “Lower Chamber” or “Bottom Chamber” refer to the chamber of the two-chamber device that is closest to the direction of gravitational acceleration. In the case of centrifugation, it is the chamber that is farthest from the axis of rotation.

As used herein, the terms “Upper Portion” or “Upper Chamber” or “Top Chamber” refer to the part of the two-chamber device that is oriented opposite to the Bottom Chamber.

As used herein, the term “Positive Fraction” refers to the portion of the mixed cell population that 1) binds to the microbubbles and 2) resides in the Upper Portion Chamber of the two chamber device following the separation procedure.

As used herein, the term “Negative Fraction” refers to the portion of the mixed cell population that 1) does not bind to the microbubbles and 2) resides in the Lower Portion Chamber of the two chamber device following the separation procedure.

As used herein, the term “Marker Group” refers to a moiety attached to a ligand, and to which a second ligand can be formulated. Multiple marker groups are suitable for use in this invention, and selection of the most suitable marker group for a given application depends upon the characteristics of the specific ligand to be used, the targeted cell type(s), and the targeting ligand conjugated to the microbubble surface. Exemplary marker groups include biotin, phycoerythrin, fluorescein, polyhistidine (His)-tag, colloidal gold, horseradish peroxidase (HRP), fluorochromes, enzymes, chelators, glycoconjugates, and avidin derivatives. In some cases, the marker group may be a domain occurring naturally on the ligand itself. For example, the constant region of an antibody may serve as a marker group; in this case, a second antibody having binding specificity for the constant domain on the first antibody may be used as a targeting ligand on the microbubble surface.

As used herein, the term “Cell” refers to an individual membrane-encapsulated unit of a living organism. Cells may comprise a unicellular organism, for example in the case of bacteria and yeast, or an individual unit of a larger organism.

### III. Microbubble Compositions

Prior art in the field of microbubble-based separation relies upon microbubble compositions previously developed for use as ultrasound contrast agents. The current invention teaches how the microbubble and separation methodology can be optimized for efficacious use as a cell separation product. We have found that, for many cell separation applications, microbubble compositions taught by the prior art result in significantly poor performance. Surprisingly, microbubble compositions that exhibit superior performance for buoyancy-based cell separation are distinct from the prior art in several key features, and in many cases said compositions would not be suitable for use as an imaging agent.

The microbubble compositions discussed below are preferentially used for separation of cells or soluble analytes using the two-chamber apparatus taught in the instant invention, although use of these compositions with other methods is also contemplated.

Considerations pertaining to microbubble compositions specifically for use in the context of cell and soluble analyte isolation are discussed, and several specific design strategies are taught. It will be obvious to one skilled in the art that these strategies can be combined or used separately in order to achieve the desired performance of the final product.

### 3.1 Optimization of Ligand Density

The strength of the adhesive bond between the microbubble and the targeted cell is a key parameter that plays a critical role in buoyancy-based cell separation. In a general sense, this bond strength is controlled by both the affinity and the avidity of the target:ligand pair. In the context of buoyancy-based separation, selection of a ligand with high affinity to the target, while maintaining high specificity, is critical. In general, it is desired to maximize the affinity of the ligand. The microbubble must be coated with a sufficient density of ligand so as to cause formation of a cell:microbubble complex within a reasonable amount of time (˜seconds to minutes). The density of the ligand on the microbubble surface should be selected so that the number of target:ligand bonds is sufficient to resist detachment of the cell from the microbubble during the buoyancy-based separation procedure. Finally, the density of the ligand on the microbubble should not be so high that cell-microbubble aggregates form. Formation of aggregates may compromise the buoyancy of the microbubble-bound cells leading to poor separation efficiency. Additionally, in some circumstances cell aggregates may persist after removal of the microbubbles, rendering the isolated cells unusable for downstream applications.

Non-specific binding of microbubbles to non-targeted is an undesirable occurrence, the likelihood of which increases with antibody density. In particular, leukocytes are expected to undergo non-specific adhesion to microbubbles. Contamination with white blood cells was in fact observed by Shi (2013), who noted that “efficient washing” may be a potential solution.

The prior art pertaining to targeted microbubbles teaches that ligand density is a key design criteria in microbubble formulation, with microbubble binding increasing with ligand density. For example, Weller (2002) synthesized microbubbles with varying concentrations of antibody ligand and demonstrated that microbubble adhesion to the intended target increased with increasing ligand density. A similar finding was reported by Della Martina (2007), using a protein-based targeting ligand. The relationship between ligand density and microbubble adhesion efficiency was further refined in Weller (2005), who taught that the adhesion strength of the microbubble is linearly proportional to the ligand density on the microbubble surface.

The prior art teaches that antibody densities on the order of 100,000 molecules per microbubble are preferred. For example, Takalkar (2004) teaches and antibody density of 100,000 per microbubble. Weller (2005), Ferrante (2009), and Tlaxca (2012) teach densities of antibody ligands of between 60,000 to 200,000 per microbubble. The ligand density may be even higher in the case of small molecules such as peptides. For example, Anderson (2011) teaches a ligand density of 800,000 per microbubble and Pochon (2010) teaches a density of 400,000 per microbubble. Shi (2013) teaches a microbubble composition comprising an antibody density of 375,000 per microbubble for buoyancy-based cell separation.

TABLE 2
Summary of ligand densities utilized in microbubble
formulations used for various applications.

| Density (molecules/ | | |
|---|---|---|
| microbubble) | Ligand Type | Reference |
| 1.0E5 | IgG antibody | Takalkar et al, J. Control |
| | | Release (2004) |
| 1.1E5 | Single-chain | Anderson et al, Invest. |
| | protein | Radiol (2010) |
| 8E5 | peptide | Anderson et al, Invest |
| | | Radiol (2011) |
| 6E4 | antibody | Weller et al, Biotech and |
| | | Bioengineering (2005) |
| 2.5E5 | glycoconjugate | Weller et al, Biotech and |
| | | Bioengineering (2005) |
| 1.1E5 | antibody | Ferrante et al, J. Control |
| | | Release (2009) |
| 2.0E5 | antibody | Tlaxca et al, J. Control |
| | | Release (2012) |
| 4E5 | peptide | Pocbon et al, Invest |
| | | Radiol (2010) |
| 3.7E5 | antibody | Shi et al, PLOS One (2013) |

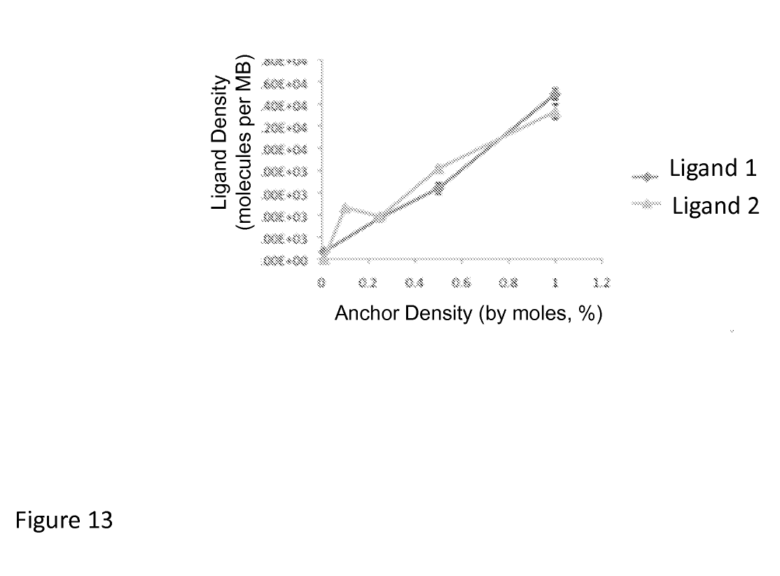

Ligand density is related to shell composition by the density of the anchor in the shell. Representative anchors are taught in Klibanov (U.S. Pat. No. 6,245,318), and include lipids and other hydrophobic molecules bearing one or more functional groups for bioconjugation of the targeting ligand. The density of the anchor is generally expressed as percent by moles of the anchor molecule relative to the total shell-forming materials. The relationship between anchor density and ligand density may be empirically determined for a given ligand and conjugation chemistry by synthesizing microbubbles bearing increasing density of anchor, reacting with excess ligand, removing unconjugated ligand, and measuring the density of conjugated ligand on the microbubbles. Exemplary methods for measurement of ligand density include ELISA and radiolabelling of the ligand and gamma counting. A representative antibody density vs ligand density plot for two representative ligands is shown in FIG. 13.

The prior art teaches a wide range of anchor density, generally between 1-20%. Rychak (U.S. Publication No. 20120244078), Hossack ((U.S. Publication No. 20140142468) both teach microbubbles comprising 2% anchor molecule, and corresponding ligand densities of 142,000 and ˜100,000 per microbubble. Klibanov (U.S. Pat. No. 6,245,318) teaches microbubbles bearing anchor densities of 7.5%; similar microbubbles were demonstrated in Villanueva (1998) and WO1999013918. Unger (U.S. Pat. No. 6,039,557) teaches a preferred microbubble formulation comprising 5% anchor density. Swenson (U.S. Pat. No. 8,293,214) teaches microbubbles comprising an anchor density of 5%.

Shi (Methods, 2013) teaches a microbubble composition comprising 7-10 mole % of anchor for use in buoyancy-based cell separation. Similar microbubbles, comprising 3% of anchor, were taught by Cuthbertson (International (PCT) Publication No. WO199055837A) for cell separation.

Microbubble formulations suitable in the instant invention comprise less than 2% anchor density, more preferably 1%. In the case of antibody used as a ligand, a density of less than 60,000 antibody molecules per microbubble, and more preferably, less than 50,000 antibody molecules, is preferred.

The prior art pertaining to targeted microbubbles for imaging and cell separation teaches microbubbles comprising ligand densities higher than those disclosed in the instant invention. Prior art also teaches that microbubble binding efficiency increases with ligand density. It would not be obvious to a skilled artisan to expect microbubbles comprising lower ligand densities to be efficacious.

Microbubbles constructed with the ligand densities specified in the instant invention will, in general, not be suitable for use as an imaging or targeted delivery agent in vivo.

The ligand density should be optimized within the range of densities specified in the instant invention in order to achieve suitable cell separation performance. Specific considerations for optimization of the ligand density are given in Table 3.

TABLE 3
Considerations for optimization of ligand density.

| Problem | Ligand Density Consideration |
|---|---|
| Cell-cell aggregation | Reduce ligand density |
| Insufficient target cell capture | Increase ligand density |
| to cells due to low target expression | |
| Insufficient target cell capture | Increase ligand density |
| due to poor ligand binding | |
| Non-specific binding of | Reduce ligand density |
| unwanted cells to microbubble | |

### 3.2. Selection of Targeting Ligand Anchor

Selection of the targeting ligand anchor is an important facet in the design of a microbubble for buoyancy-based separation. Anchors suitable for use in the instant invention comprise a hydrophobic portion, providing for insertion into the lipid shell; a hydrophilic portion, which is in contact with the liquid media; and a conjugation residue, providing for linkage of the targeting ligand to the anchor. The anchor may be incorporated into the shell upon synthesis of the microbubbles, or may be inserted into the intact microbubble after preparation. The anchor may be modular, comprising separate hydrophobic, hydrophilic, and conjugation portions, or may comprise a single entity. Representative anchors suitable for use in the instant invention are disclosed, for example by Klibanov ((U.S. Pat. No. 6,245,318).

Conjugation residues comprising biocompatible conjugation chemistries are preferred. Suitable residues are disclosed, for example, by Klibanov ((U.S. Pat. No. 6,245,318) and Unger ((U.S. Pat. No. 6,139,819). Maleimide, protected sulfhydryl, amine, and carboxyl functionalities are preferred.

Anchors in which the hydrophilic portion comprises a polymer chain are preferred. In a preferred embodiment, the polymer chain is polyenthyleneglycol (PEG). In one embodiment the average molecular weight of the PEG is between 500-5,000. In a more preferred embodiment, the average molecular weight of the PEG is between 1,000-4,000.

Examples of hydrophobic moieties suitable for use in the anchor of the instant invention include branched and unbranched alkyl chains, cyclic compounds, aromatic residues and fused aromatic and non-aromatic cyclic systems. In some instances the hydrophobic moiety will consist of a steroid, such as cholesterol or a related compound. Preferred species include lipids, steroids, and hydrophobic polyamino acids.

In all cases, it is desired that the hydrophobic portion of the anchor remain firmly within the microbubble shell during the process of cell separation. In some cases, buoyancy based cell separation will require the cell-microbubble complex to experience motion (e.g., movement in the direction normal to applied gravity); this motion introduces a force on the components joining the cell and microbubble together microbubble. Under some circumstances, said force may be sufficient to remove the hydrophobic portion of the anchor from the microbubble shell, thereby releasing the cell from the microbubble before separation is completed. This would be undesirable.

Unwanted anchor detachment from the microbubble shell may be avoided by selecting anchors in which the anchor is firmly bound within microbubble shell. Short range attractive forces between the hydrophobic portion of the anchor and hydrophobic tails of adjacent shell-forming lipids are assumed to be the mechanism for maintaining the anchor in the microbubble shell. The anchor can be selected so as to optimize the strength of these attractive forces for a given shell composition. This can be achieved, for example in the case of lipophilic hydrocarbon chains, by selecting species with a sufficient number of carbon atoms to form a sufficient number of bonds with adjacent shell lipids. Preferred compositions comprise between 14 and 24 carbon atoms. More preferred compositions comprise between 16 and 20 carbon atoms. The number of lipophilic chains can be varied, and will generally comprise one or two hydrocarbon chains.

In some cases, it may be desirable for the anchor molecule to be removed from the microbubble shell after cell separation is completed. For example, in some cases it would be desirable to remove the microbubble from the selected cell, leaving only the ligand-anchor attached to the cell. This renders the cell non-buoyant and also removes much of the microbubble shell material, the presence of which may induce artifacts on downstream assays (e.g. flow cytometry).

Removal of the anchor from the microbubble shell may be achieved by, for example, applying a positive pressure to the microbubble. In this case, the anchor molecule may be selected so that it is ejected from the microbubble shell upon compression or collapse of the microbubble. Anchors suitable in this respect generally comprise those in which the hydrophobic portion of the anchor exists at a phase different from that of the remainder of the microbubble shell during the microbubble compression. Preferred embodiments for use in a shell comprising lipids in the condensed phase comprise anchors in which the hydrophobic portion is in the liquid expanded phase. Exemplary anchor molecules for use in this respect include functionalized fatty acid PEG derivatives, and functionalized PEG lipids. Preferred anchors include those in which the hydrophobic portion comprises single chain fatty acids, particularly stearic acid and palmitic acid. Preferred embodiments include anchors in which the hydrophilic portion comprises PEG of average molecular weight between 500 and 5,000.

Ejection of the anchor may be accompanied by microbubble collapse, although this is not necessary.

Ejection of the anchor may also be achieved by applying a negative pressure to the mixture of cell-microbubble complexes. Similar considerations to anchor selection apply in this case.

Microbubbles in which the shell comprises between 0.1 and 1% by moles of the anchor are preferred in the instant invention.

### 3.3. Microbubble Shell Considerations

### 3.3.1 Requirement for Biocompatibility and Downstream Assay Compatibility

The shell of a microbubble suitable for buoyancy-based separation should be composed of biocompatible and bioinert materials. The shell materials should be inert to the biological substances being separated, and not interfere in downstream assays. For example, the anionic lipid phosphatidylserine taught by Rongved ((U.S. Publication No. US20070036722) would not be suitable for use in the instant invention. Phosphatidylserine is known to be a marker of cell death, and unwanted adhesion of the microbubble to phagocytic cells present in the mixed cell population (for example, blood, PBMC, spleen, or bone marrow) may occur. Similarly, the presence of phosphatidylserine may interfere with downstream assessment of the isolated cells. For example, AnnexinV is commonly used as a marker for apoptic cells in flow cytometry, and the presence of phosphatidylserine contributed from the microbubble shell may bind AnnexinV and lead to artifactual assay results. Other lipids commonly taught in the context of microbubble shell (for example, phosphatidic acids, phosphatidylinositol, cardiolipins, sphingomyelins) participate in cell signaling, and are therefore generally unsuitable for use in the instant invention.

Specific shell materials meeting the requirement for biocompatibility are discussed in the sections below.

### 3.3.2 Reduction of Surface Charge

The deliberate inclusion of shell materials that contribute a surface charge to the microbubble used in cell separation is taught, for example by Mattrey (International (PCT) Publication No. WO2009052057), Simberg (2009), Rongved (U.S. Publication No. US20070036722), Cuthbertson (U.S. Publication No. US20030104359), and Kulseth (U.S. Pat. No. 6,806,045). This is consistent with the prior art from microbubbles developed as imaging reagents, in which the presence of a surface charge was desired in order to stabilize the intact microbubble, avoid microbubble fusion, and in some cases avoid or to aid in electrostatic binding to cells or other biological substances. For example, Klaveness (U.S. Pat. No. 6,261,537) teaches microbubbles in which 75% or more of the microbubble shell constituents are charged. Unger (U.S. Pat. No. 6,139,819) similarly teaches a preferred microbubble formulation comprising a mixture of anionic, neutral, and stabilizing shell components.

We have made the surprising discovery that, by careful selection of shell components, microbubbles bearing low to essentially no net surface charge can be prepared in large numbers, with excellent stability, and which exhibit negligible non-specific adhesion to cells in the context of cell separation. Furthermore, in the context of buoyancy-based cell separation, the presence of surface charge (positive or negative) on the microbubble is not desirable. Surface charge constitutes a basis for electrostatic interactions between the microbubble and biological components, potentially leading to undesirable non-specific binding to the microbubble. Thus, selection of shell materials comprising predominantly species with no net charge is preferred.

In some embodiments, greater than 50% of the lipids forming the shell present no net charge. In a preferred embodiment, greater than 75% of said lipids present no net charge. In a most preferred embodiment, greater than 85% of said lipids present no net charge.

Exemplary shell forming lipids that present no net charge include phosphatidylcholines, in particular disteroylphosphatidylcholine, dipalmitoylphosphatidylcholine, and dimyrstylphosphatidylcholine), disteroylphosphatidylethanolamines, fatty acids, in particular stearic acid and palmitic acid, PEGylated ceramides, and PEGylated fatty acids.

In some cases, inclusion of lipids containing a net surface charge may be unavoidable. For example, when using anchor molecules comprising DSPE-PEG (which bears a negative charge). In these cases, it is desirable to reduce the overall amount of charged lipids to as low as possible. In the case of unavoidable surface charge, use of a second surfactant comprising a PEG (of average molecular weight 1,000-5,000) and in a density of greater than 1% of the total lipid content may be used to further reduce non-specific cell binding to the microbubble surface by providing a stearic barrier.

### 3.3.3 Requirement for Stability During Separation Procedure

The shell of a microbubble suitable for buoyancy-based separation should be resistant to collapse during the separation procedure. That is, the shell should remain intact and prevent the release of the gas core or reduction of buoyancy of the microbubble. This can be achieved, for example, by selecting shell components able to retard the motion of the encapsulated gas through the shell. Microbubbles described in the prior art do not necessarily have this feature. For example, Shi (2013) prepared antibody-conjugated microbubbles for use in buoyancy-based separation and noted that the main limitation to the technique was instability of the microbubble in blood. The instant invention teaches compositions that overcome this deficiency.

The microbubbles comprising instant invention must be able to remain intact (i.e., not undergo irreversible collapse) under the range of hydrostatic pressures applied during the buoyancy-based cell separation process. The range of these pressures can be assumed to be equal to the range of pressures compatible with the biological materials being separated. In the case of cells, said pressures are generally applied in the context of centrifugation, expressed in terms of relative centrifugal force (RCF). Microbubbles able to resist degradation upon centrifugation at a RCF of up to 500×G are preferred in the instant invention.

### 3.3.4 Microbubble Collapsibility

Collapse of the microbubble represents an attractive method for removing the microbubbles from the targeted cells after the isolation procedure. This is of particular relevance for positive selection applications. Microbubble collapse, as used here, renders the cells no longer buoyant, allowing them to be concentrated, buffer exchanged, and pipetted using standard laboratory methods. Collapse of the microbubble also opens the possibility of sequential separation, as discussed elsewhere in the proposal. Finally, collapse of the microbubble provides a means for removing the microbubble shell and gas components from proximity to the targeted cells, which is desirable in the context of returning cells to their native or “untouched” state as quickly as possible.

Collapse is defined here as reducing or eliminating the buoyancy of the microbubble, and is accompanied by an alteration in the structure that the lipids comprising the microbubble shell take. Microbubble collapse may be achieved by either condensing the encapsulated gas to a liquid, or by releasing the encapsulated gas from the microbubble into the surrounding liquid. Collapse behavior under conditions suitable for use in buoyancy-based cell separation may be engineered into the microbubble by careful selection of the shell or gas components.

In is generally desirable that microbubble collapse be achieved rapidly, within several seconds.

A key aspect of the microbubble collapse process is that it be implemented so as to minimize perturbation to the cells. That is, the procedure should not expose the cells to adverse conditions or otherwise alter the cells in manner undesirable for their subsequent use. For example, reduction in viability due to the cell separation procedure is generally not desirable for cells to be used in downstream functional assays. Alteration in cell surface proteins is generally not desirable for cells to be assessed in downstream staining-based assays such as flow cytometry. Alteration in transcription is generally not desirable for cells to be used in downstream messenger assays such as PCR. Alteration in activation state is generally not desirable for cells to be used downstream in a therapeutic context, for example in cell-based therapy.

In a preferred embodiment, microbubble collapse occurs under conditions compatible with biological substances, including cells, proteins, nucleic acids, and other biomolecules. In a preferred embodiment, collapse occurs at physiological pH. In a preferred embodiment, collapse occurs at temperatures between 2-39 degrees C. In a preferred embodiment, collapse occurs in an isotonic medium. In a preferred embodiment, collapse occurs without the use of detergents or reagents that may alter cell membrane integrity or cellular homeostasis.

Methods requiring changes in salt content, pH, or temperature beyond physiologically tolerable levels, for example as taught in Cuthbertson (U.S. Publication No. US20030104359), are not compatible with the instant invention. Use of detergents, (as taught by Jablonski U.S. Pat. No. 8,813,586), enzymes (as taught by Kulseth U.S. Pat. No. 6,806,045), and hydrolysis (as taught by Cuthbertson, U.S. Publication No. 20030104359) are similarly not desirable in the context of the present invention.

Use of ultrasound at high mechanical index as a method for removing microbubbles from cells, as taught by Toma (U.S. Pat. No. 8,640,269) are not compatible with the instant invention. High MI ultrasound is known to cause sonoporation, which would be undesirable in the context of isolating unperturbed cells.

In designing a collapsible microbubble, it is desirable to enable collapse to occur when desired (i.e., after separation is complete), for example as a method to remove the microbubbles from the bound and selected cells. Collapse may be triggered by a number of actions applied by the user. In general, these actions will alter the arrangement of lipids in the microbubble shell, resulting in structures other than the monolayer of the intact microbubble.

A preferred method for achieving microbubble collapse is the application of pressure to the microbubble shell. This may be easily achieved by the application of hydrostatic pressure, for example in the two-chamber device of the instant invention. Other methods included depressing the plunger in a closed syringe, use of a vacuum chamber, or rapidly forcing the cell:microbubble dispersion through a syringe needle.

A second method for achieving microbubble collapse is through diffusion of the gas core through the lipid shell into the surrounding medium. This can be achieved, for example, by immersing the cell-microbubble complexes into a buffer in which the partial pressure of the encapsulated gas is essentially zero. This creates a gradient across the shell of the microbubble, leading to collapse of the microbubble. As discussed elsewhere in the specification, the contents of the gas core can be selected so as to enable microbubble collapse in the instant invention.

3.3.5 Absence of Residual Shell Components after Microbubble Collapse

It is desirable to remove as much of the shell from the targeted cell as possible.

Although Kulseth (U.S. Pat. No. 6,806,045) teaches that a proportion of microbubble encapsulating material may remain on the cell after microbubble collapse, this is not desirable in the context of the instant application because minimizing perturbation to the cells is specified. Additionally, the presence of microbubble shell components residual on the targeted cell may actually diminish the efficacy of buoyancy-based separation due to aggregation of the targeted cells.

We have found that, in certain undesirable situations, the residual shell formed after microbubble collapse may take the form of tubes or discs several (1-10) micrometers in size. Such structures, when retained on the surface of the cells, may interfere with downstream assays; in the case that the anchor and ligand is retained within the collapsed shell, unwanted cell-cell aggregation may occur. In extreme cases, this can cause formation of aggregates comprising hundreds of cells, rendering the separated cells unsuitable for downstream use.

We have made the surprising discovery that the degree of residual shell attached to the targeted cell can be modulated by careful selection of the shell materials and the collapse procedure. In a preferred embodiment, it is desirable for the microbubble shell to take the form of multiple sub-micron particles after collapse. The formation of large (micron-scale) structures capable of bridging cells and forming aggregates are not desired.

### 3.3.6 Modulation of Collapsed Microbubble Fragment Size by Using Differential Shell Phase Transition Temperature

In one embodiment, the physical phase of the shell forming materials can be selected so as to preferentially cause the collapsed microbubbles to predominantly take the form of sub-micron particles. Without being bound to any particular theory, the structure that the collapsed microbubble shell forms is related to the physical phase of the lipids during the collapse procedure. Specifically, the inclusion of shell forming lipids that are predominantly in the liquid expanded phase during the collapse procedure are preferred. Upon collapse, lipids predominantly in the liquid expanded phase tend to associate into structures of less than 1 micron in the longest dimension. In contrast, lipids that are in the condensed phase during the collapse procedure tend to form tubes, folds, sheets, and other large (1 um or greater) structures.

An efficacious method of ensuring that the lipids comprising the microbubble shell are in the liquid expanded form is by heating the microbubble:cell solution past the main transition temperature of the lipid. In the context of the instant invention, it is critical that this temperature be within physiologically acceptable limits for biological samples, herein defined as 2-38 degrees Celsius.

In one embodiment of the invention, the separation process, comprising incubation of the cells with the microspheres, separation, and collection of the separated fractions, occurs at one temperature and the microbubble collapse process occurs at a second, higher temperature.

In one embodiment of the invention, the separation process occurs at room temperature (20-25 degrees Celsius) and the microbubble collapse process occurs at 35 degrees Celsius.

In one embodiment of the invention, the separation process occurs at on ice or under refrigeration (4-15 degrees Celsius) and the microbubble collapse process occurs at room temperature.

Heating the cell-microbubble suspension to the higher collapse temperature can be accomplished using any number of routine laboratory methods, including placing the suspension in a cell incubator, placing in a heated water bath, or placing in a heat block.

Practice of this aspect of the instant invention requires use of shell-forming materials that have a phase transition temperature in the desired range (approximately 4-35 degrees Celsius). In a preferred embodiment, the microbubble shell comprises a combination of low-transition temperature and higher transition temperature (e.g., greater than 38 degrees Celsius) lipids, selected in a ratio such that the overall melting point of the microbubble shell is at the desired temperature range. Thus, microbubbles are doped with a second shell forming material with the intent of depressing the phase transition temperature of the lipid monolayer.

Several shell-forming materials are suitable for reducing the melting temperature of the microbubble shell. Lipids that are suitable for use as low melting temperature species include saturated phosphatidylcholine comprising 13, 14, or 15 acyl chains, and saturated phosphatidylethanolamine of 12 acyl chains. Some unsaturated, mixed acyl phospholipids, and lysolipids are also suitable for use in this context.

TABLE 4
Lipid species suitable for use in reducing the phase
transition temperature of the microbubble shell.

| | Transition |
|---|---|
| Lipid Species | Temperature (deg C.) |
| 13:0 phosphatidylcholine | 14 |
| 14:0 phosphatidylcholine | 24 |
| 15:0 phosphatidylcholine | 35 |
| 12:0 ethanolamine | 29 |
| 18:19 phosphatidylcholine | 12 |
| 14:0-16:0 phosphatidylcholine | 35 |
| 16:0-14:0 phosphatidylcholine | 27 |
| 18:0-14:0 phosphatidylcholine | 30 |
| 18:0-18:1 phosphatidylcholine | 6 |
| 16:0-18:1 phosphatidylethanolamine | 25 |

In one embodiment of the invention, between 50 and 90% of the shell forming materials, by moles, have a phase transition temperature between 0-35 deg C. The remaining shell materials should have a phase transition temperature of greater than 38 degrees C.

In one embodiment of the invention, between 1 and 40% of the shell forming materials, by moles, have a phase transition temperature between 0-35 deg C. The remaining shell materials should have a phase transition temperature of greater than 35 degrees C.

It will be clear to one skilled in the art that microbubbles comprising lipids of low melting temperature will not be suitable for use as an imaging agent or for any in vivo application in which the temperature of the subject is 38 deg C or greater.

The inclusion of polymer-grafted shell materials can also be used to depress the phase transition temperature to the desired range. PEG-grafted phospholipids, such as disteroylphosphatidylethanolamine, are especially preferred in this respect. The magnitude of the reduction in phase transition temperature is proportional to the average molecular weight of the PEG chain and to the density of the PEG-lipid in the microbubble shell, and both parameters may be varied independently to achieve the desired phase transition temperature. The use of polymer-grafted shell materials may be used in conjunction with lipids of reduced phase transition temperature.

In one embodiment of the invention, between 1 and 15% of the shell forming materials, by moles, comprise a PEG-grafted lipid. The remaining shell forming materials have a phase transition temperature of between 0-38 degrees C.

On one embodiment of the invention, between 1 and 15% of the shell forming materials, by moles, comprise a PEG-grafted lipid. The remaining shell forming materials comprise at least one shell forming material having a phase transition temperature of between 0-38 deg C.

It should be noted that the aspect of the invention comprising the selection of shell components designed to be in the liquid expanded phase on collapse can be achieved with any encapsulated gas taught in the prior art. That is, practice of this aspect of the invention is not limited to gases that also exhibit phase change behavior over the prescribed region.

### 3.3.7 Modulation of Collapsed Microbubble Fragment Size by Using Differential Shell Solubilit

In another embodiment of the invention, the size of the shell fragments formed after crushing can be modulated by the inclusion of a shell component that is essentially soluble in water. Without wishing to be bound by any particular theory, it is believed that during the microbubble collapse procedure, the shell component of high water solubility is released from the shell and able to dissolve into the surrounding aqueous buffer, leaving residual shell components of low water solubility to form structures of reduced size, preferably less than 1 um in the longest dimension.

It should be noted that modulation of the temperature during microbubble collapse is not required for application of this aspect of the instant invention rather, this invention can be practiced with microbubbles over a range of temperatures that are compatible with biological substances.

In some cases, the shell comprises a first surfactant and a second surfactant having higher water solubility than said first surfactant.

In a preferred embodiment, said second surfactant comprises between 15 to 50%, by moles, of the microbubble shell.

In some cases, the first surfactant of the two-component shell is selected from the group consisting of dilauroylphosphatidylcholine, dimyristoylphosphatidylcholine, dipalmitoylphosphatidylcholine, disteroylphosphatidylcholine, diarachidoylphosphatidylcholine, dibehenoylphosphatidylcholine, dilignoceroylphosphatidylcholine, and other phospholipids bearing no net headgroup charge.

In some preferred cases, the first surfactant is disteroylphosphatidylcholine.

In some cases, the second surfactant is selected from the group consisting of fatty acids, PEG-lipids, and PEG-fatty acids.

In some cases, the second surfactant of a two-component amphipathic shell is a polyethylene glycol ester of stearic acid.

### 3.4. Microbubble Gas Core Considerations

Prior art in the field strongly discourages the use of water-soluble gasses in the fabrication of microbubbles. Indeed, Cuthbertson (0028) states that perfluorocarbons and other gases that form “highly stable” microbubbles are preferred. Similar teaching is found in Rongved. In some cases, however microbubble collapse and modulation of the residual shell fragment size can be modulated by careful selection of the solubility of the encapsulated gas. In this embodiment, gases that have moderate to high solubility in aqueous buffers are preferred. Upon exposing the gas core to the surrounding aqueous media upon microbubble collapse, dissolution of the gas core into the surrounding media occurs, rendering collapse of the microbubble complete.

In one embodiment of the present invention, gasses that have moderate to high solubility in aqueous solvents and exhibit biocompatibility are preferred. Air, oxygen, carbon dioxide, and nitrogen are especially preferred. Perfluorohexane and perfluoropentane are exemplary gases with extremely low water solubility not suitable for use in this aspect of the instant invention.

In this embodiment, use of shell-forming materials that retard movement of the gas across the shell are preferred in order to ensure stability of the microbubble during the separation procedure. Shell forming materials comprising lipids with long hydrophobic chains are specifically preferred. In a preferred embodiment, lipids comprising greater than 18 acyl chains, more preferably greater than 20, are preferred.

In all cases, it is desirable that gas core of the intact microbubble is predominantly in gaseous form at 4-37 degrees Celsius.

In some cases, the gas core is selected from the group consisting of air, nitrogen, oxygen, carbon dioxide, or mixtures thereof.

In some preferred cases, the gas core is nitrogen or nitrogen admixed with an osmotic gas modifier.

In some preferred cases, the gas core is air or air admixed with an osmotic gas modifier.

Synthesis of microbubbles containing gases with moderate to high solubility present some challenges from a manufacturing standpoint. For example, in the case where ligands are conjugated to the surface of intact microbubbles, stability on the order of hours to days may be required before microbubbles are packaged and ready to use. This problem may be avoided by synthesizing microbubbles using a gas of low solubility, and subsequently exchanging the gas in the final packaging step. For example, microbubbles may be synthesized with a gas core comprising decafluorobutane, processed and prepared for packaging, and the gas core replaced with air or other moderate to high solubility gases taught in this invention.

Lyophilization and other drying methods provide preferred methods for exchange of the gas core. For example, microbubbles containing a low solubility gas can be synthesized, packaged into vials, and lyophilized. After lyophilization, the vials are evacuated and replenished with a headspace comprising a second gas of moderate to high solubility. The vials are then sealed under said second gas. Upon reconstitution with aqueous buffer, microbubbles containing the second gas are formed.

### 3.5 Microbubble Diameter

The magnitude of the buoyant force offered by the microbubble is proportional to the volume of the gas core of the microbubble, and hence to the microbubble diameter. Efficient buoyancy-based cell separation, in which cells are isolated with high yield and purity and with minimal perturbation, can be achieved by selection of microbubbles of the optimal diameter.

Prior art teaches that microbubbles of relatively large diameter are preferred for buoyancy-based separation. For example, Shi (2013) teaches that microbubbles of large diameter are to be preferred, and that a microbubble of diameter 9 um is sufficient for isolating a single cell. Shi (2013) presents an analytical framework relating the diameter of the microbubble to the number of microbubbles required to lift a single cell, and teaches that for a 3 um microbubble approximately 20 microbubbles are required in order to buoyantly separate a cell. Unexpectedly, we have found that by following the methods disclosed in the instant invention, cells can be isolated by far fewer microbubbles, and using a significantly lower antibody density than taught by Shi. For example, we demonstrate in Specific Example 27 that cells can be buoyantly isolated using a microbubble formulation of an average diameter of ˜3 um with between on average fewer than 10 microbubbles per cell.

In one embodiment of the invention, the average diameter of microbubbles used for buoyancy-based cell separation are of diameter 3 um, or more preferably 4 um, or more preferably 5 um. In each case, 50% of the total microbubble number are within 1 micron of the specified mean diameter.

In some cases, use of large microbubbles (of diameter 5 um or greater) may be undesirable. For example, when using such microbubbles for positive selection of cells and removing the microbubbles by collapse, the collapse procedure may create a tension on the cell membrane, leading to undesirable bioeffects, including loss of viability, on the selected cells. This may be avoided by using small microbubbles, which have a smaller footprint when bound to cells and which therefore may induce a much reduced membrane tension upon microbubble collapse.

For example, when performing buoyancy-based separation of rare cells (<1% of the total cell population), small microbubbles may be preferable due to 1) the enhance diffusive motion relative to larger microbubbles, and 2) the ability to achieve a higher microbubble to cell ratio in a given volume relative to large microbubbles.

In this embodiment, the average diameter of microbubbles used for buoyancy-based cell separation are of diameter 2 um, or more preferably 1 um. In each case, 50% of the total microbubble number is within 1 micron of the specified mean diameter.

In this embodiment, a higher number of microbubbles, between 5 to 50, may be required to bind to a given cell in order to confer sufficient buoyancy.

### 3.6. Selection of Targeting Ligand

The prior art provides little guidance in terms of selection of targeting ligand for buoyancy-based cell separation. In the context of the instant invention, ligands exhibiting high specificity (<1% non-specific binding), high affinity (preferably Kd in the nM range), and biocompatibility are preferred. Antibodies, preferably monoclonal antibodies, are suitable for most applications of the instant invention.

In some embodiments, humanized antibodies are preferred. For example, when the positively selected cells are subsequently administered to a human patient.

In some cases it may be desirable to use ligands exhibiting a significantly lower affinity and molecular weight. For example, the use of ligands having an affinity of at between 10 to up to 100 times lower than most antibodies, and a molecular weight of no greater than 50 kDa, is desirable in one embodiment of the invention. Peptides, glycoconjugates, aptamers, single-chain antibodies, and Fab fragments are suitable in this regard.

The use of small, low affinity ligands is advantageous as a method for achieving complete release of the ligand from the targeted cell upon microbubble collapse. In this case, binding of a large number of ligands to the targeted cell is required to effect separation, and in this case high ligand densities may be warranted. A higher site density of ligand is achievable in this case due to the reduced molecular weight of the ligand.

### IV. Methods

In one aspect, the invention provides a method of separating target cells from a mixed population in a liquid sample, the method comprising the steps of i. mixing the mixed population with one or more microbubble compositions taught in the instant invention, ii. incubating the liquid sample at a temperature between 4° C. and 37° C. for a sufficient time to allow the target cells and the microbubbles to form cell-microbubble complexes; iii. transferring the liquid sample to the two chamber apparatus; iv. applying sufficient centrifugal force to the liquid sample containing the cell-microbubble complexes for a sufficient period of time to cause the cell-microbubble complex to become enriched in the upper portion of the container, and the remaining cell population to become enriched in the bottom portion of the container; iv. Collecting the buoyant and sedimented cell populations; v. exerting sufficient pressure to the buoyant fraction to collapse the microbubbles, thereby liberating the target cell from the microbubble-cell complex; and vi. collecting the target cells.

In one aspect, the invention provides a method of separating target cells from a mixed population in a liquid sample using a two chamber apparatus, the method comprising the steps of: i. mixing the cells with a buoyant microbubble composition in the liquid sample, ii. incubating the liquid sample at a temperature between 4° C. and 37° C. for a sufficient time to allow the target cells and the microbubbles to form cell-microbubble complexes; iii. transferring the liquid sample to the two chamber apparatus, iv. applying sufficient gravitational force to the liquid sample containing the cell-microbubble complexes in said two-chamber apparatus for a sufficient period of time to cause the cell-microbubble complex to become enriched in the upper chamber of said two-chamber apparatus, and the remaining cell population to become enriched in the bottom chamber of said two-chamber apparatus; v. collecting the cells from each chamber, and vi. exerting sufficient pressure to the buoyant cells to collapse the microbubble, thereby liberating the target cell from the microbubble-cell complex; and vii. collecting the target cells.

The two chamber apparatus disclosed here is suitable for use in positive selection, negative selection, and depletion.

In some cases, the time period for incubating the liquid sample is between 0.1 and 60 minutes.

In some cases, the time period for applying centrifugal force to the liquid sample is between 0.1 and 60 minutes.

In some cases, the relative centrifugal force applied is between 1 and 500.

In some cases, the pressure that is exerted on the top chamber of the apparatus is in the form of hydrostatic pressure and is applied by decreasing the volume of the top chamber of the apparatus by depressing a plunger.

The method and apparatus disclosed herein provides for separation of target cells to a high degree of purity.

### Centrifugation and Collection of Buoyant Particles

One aspect of the invention is the method used to effect separation of target cells from the bulk. It is preferable to perform the separation procedure in as little time as possible, in order to minimize the possibility of cell death, activation, or other changes to the cell. To this end, methods that accelerate buoyancy-based separation are to be preferred. Centrifugation and other means of increasing the gravitational force are preferred, as effective separation can be completed within seconds to minutes. The magnitude of the centrifugal force will generally be dictated by the requirement that cells not be damaged by the procedure. Thus, centrifugation at below 500×G, and more preferably at or below 300×G, are preferred. Additionally, a robust means of collecting both the microbubble-bound cells and the sedimented cells is required. Finally, it should be noted that removal of the non-targeted cells from the positive fraction is required to achieve a high degree of purity. This feature is desirable in the context of both positive and negative selection applications. This is especially important when using this technology to isolate rare cells, as contamination of the positive fraction with even a small number of unwanted non-targeted cells can greatly diminish the resulting purity.

Simberg (2009) and Shi (2013) both teach that standard centrifugation in a single-chamber (such as an Eppendorf tube) can be used to cause microbubbles and microbubble-bound cells to form a floating layer. However, the authors (Simberg, p. 395) note that collection of the buoyant layer formed using their system was technically challenging, and this difficulty ultimately prevented them from directly assessing the microbubble-bound cells. Shi (2013) utilizes an inverted conical tube, the tip of which has been removed to enable collection of the floating microbubble cake. It will be apparent to one skilled in the art that collection of the sedimented fraction (in this case, pelleted onto the lid of the inverted conical tube) will be difficult or impossible. Shi (2013) observed passive entrapment of unwanted cells in the microbubble (buoyant) fraction, and noted that more efficient washing steps are warranted. The two-chamber device of the current invention provides a means for facilitating direct collection of the buoyant fraction, collection of the sedimented fraction, and for minimizing non-specific carryover of non-buoyant cells into the buoyant fraction.

A key aspect of the difficulty in collecting the buoyant, microbubble-bound cell fraction stems from the fact that this fraction behaves substantially as a foam and is resistant to collection using standard laboratory methods such as pipetting. Although it is possible to carefully suction off the floating cake, this is a time-consuming and error-prone procedure and is not suitable for use in a commercial product. The invention described herein overcomes this difficulty with by use of the two-chambered apparatus.

Rongved and Cuthbertson each teach that centrifugation (0041) followed by “decantation, transfer from one syringe to another, or simply skimming off the floating microbubble layer” is a method for collecting the microbubble-bound cell fraction. However, experience demonstrates that these methods for collection of the microbubble-bound cell fraction suffer from low efficacy. Skimming, as observed by Simberg (2009), is technically difficult, and time consuming; Cuthbertson demonstrated that this method results in a purity of 50-87% (0110 and 0160), which is too low for practical use. Likewise, decantation (“pouring off” of the microbubble fraction) results in low purity, as unwanted cells in the cell pellet become entrained with the collected fraction. This was observed by Shi (2013), who noted contamination of the positive fraction with leukocytes. The apparatus and methods disclosed herein enable the user to cleanly isolate the buoyant fraction from the negative pellet, and easily collect the desired cells.

Devices for isolating buoyant microbubbles during centrifugation have been taught in the context of microbubble purification and washing. For example, Rychak et al (2006) teaches that soluble components (for example, unbound antibody or similar) can be removed from microbubbles by centrifuging in a syringe/stopcock apparatus. The plunger is removed from the syringe, and a closed stopcock is placed at the neck. The device is placed upright and centrifuged, resulting in the formation of a cake consisting of most of the microbubbles. The infranatant, containing the soluble components, can be removed by slightly opening the stopcock to enable the liquid fraction to be slowly removed without disturbing the cake. Such a system, in which the non-buoyant fraction is removed through a stopcock or other narrow opening, is not practical for removing cells or other solid particles to a high degree of purity. This is because centrifuged cells aggregate at choke points (for example, the neck of the syringe), necessitating multiple rounds of centrifugation to remove the unwanted cells to a useful degree of purity. The two-chamber apparatus does not require valves or other constrictions through which cells must pass; instead, a tapered insert with an opening many times greater than the cell dimension serves as a barrier between the upper and lower chambers, each comprising the buoyant and sedimented fractions, respectively.

It will be apparent to one skilled in the art that existing barrier-type tubes, for example LeucoSep™ (Greiner Bio One), SepMate™ (StemCell Technologies) and tube apparatus designed for gradient-type separations, are not suitable for use in the context of the instant invention. Collection of the sedimented fraction is not feasible with these devices. Moreover, only one loading orientation is possible (mixed cells in upper fraction). This requires that negative cells sediment into the bottom chamber in order to effect separation, which in the case of a rare population of targeted cells may compromise purity. The two-chamber device of the instant invention makes collection of both be positive (buoyant) and negative (sedimented) fractions possible, and also enables the flexibility to optimize the loading protocol for a given application.

In a particularly surprising feature of the two-chamber device, concentration of cells and cell-bound microbubbles into a pellet and cake, respectively, is not necessary. Rather, it is sufficient to simply cause the cells to migrate into the desired chamber (for example, using applied centrifugal force) without pelleting or caking. This has potential advantages in that cells remain in solution and are not exposed to a gas-liquid interface, as may happen during caking.

The two chamber apparatus disclosed here is suitable for use when the material to be separated comprises cells, including blood, splenocytes, bone marrow, leukapheresis product, bacteria, or tissue homogenate. It is also suitable for use when the material to be separated comprises soluble analytes.

It will be clear to one skilled in the art that the buffer in the upper and lower chamber does not necessarily have to be identical. For example, test sample dispersed in FACS buffer may be loaded into the top chamber, and the bottom chamber loaded with normal saline. During the separation procedure, the non-targeted cells accumulate in the bottom chamber and are subsequently collected (negative selection). This feature provides an especially convenient method of achieving buffer exchange, and in the example discussed above provides a method for limiting contamination of the negatively selected cells with non-buoyant contaminants that may be present in the test sample.

### Design of the Two-Chamber Apparatus

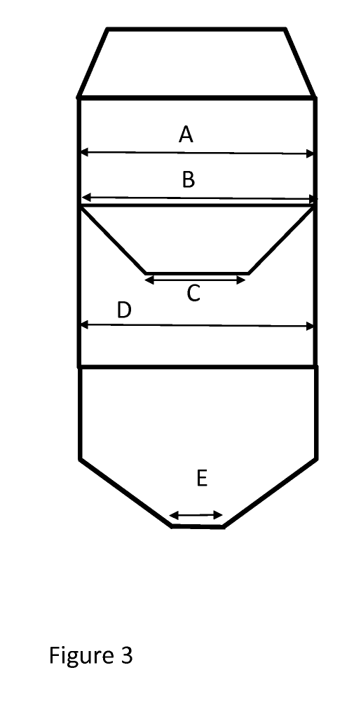

The dimensions of the two-chamber apparatus taught here can be altered to suit a wide variety of applications in buoyancy-based separation. For example, the ratio between the narrow insert diameter (measurement C, FIG. 3) and the upper chamber diameter (measurement A, FIG. 3) may be tuned so as to prevent leakage of fluid from the upper chamber into the bottom chamber during loading of the upper chamber. For example, the diameter of the tapered end of the bottom chamber (measurement E, FIG. 3) can be set so as to achieve suitable concentration of pelleted cells for the desired application. For example, the overall dimensions of the apparatus can be selected so as to encompass a desired volume. For example, the dimensions of the apparatus can be selected so as to fit into a conical tube or centrifuge bucket.

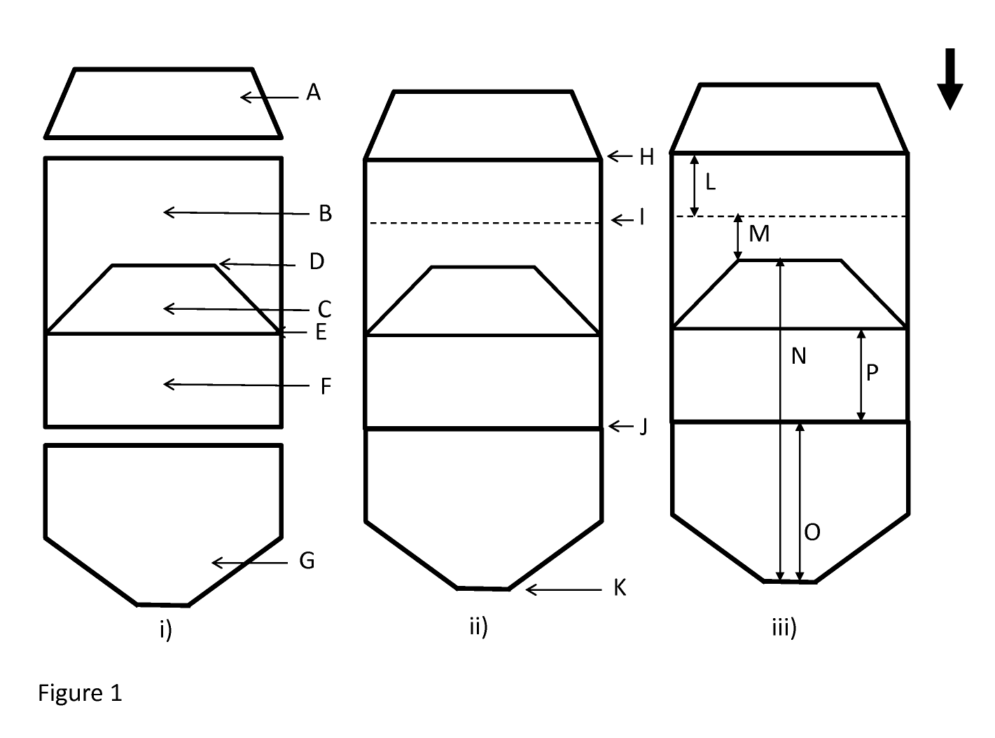

In one embodiment, the distance between the insert and the air-liquid interface (distance M in FIG. 1.iii) is maximized and the distance from the insert to the bottom of the bottom chamber (distance N) is minimized. The Test Sample is loaded into the bottom chamber, and collection buffer into the upper chamber. Separation is effected by gravity or centrifugation for a sufficient time such that substantially all of the buoyant particles (comprising microbubbles and cell-microbubble complexes) have moved into the upper chamber. Separation may be halted and the upper and lower samples collected before the buoyant samples reach the air-liquid interface. This embodiment may be advantageous in the case when contact between microbubbles and attached cells with air is not desired, for example in the case of cells sensitive to air. This may also be desirable in the case when close packing of the positively selected cells is not desirable, for example to reduce the possibility of aggregation.

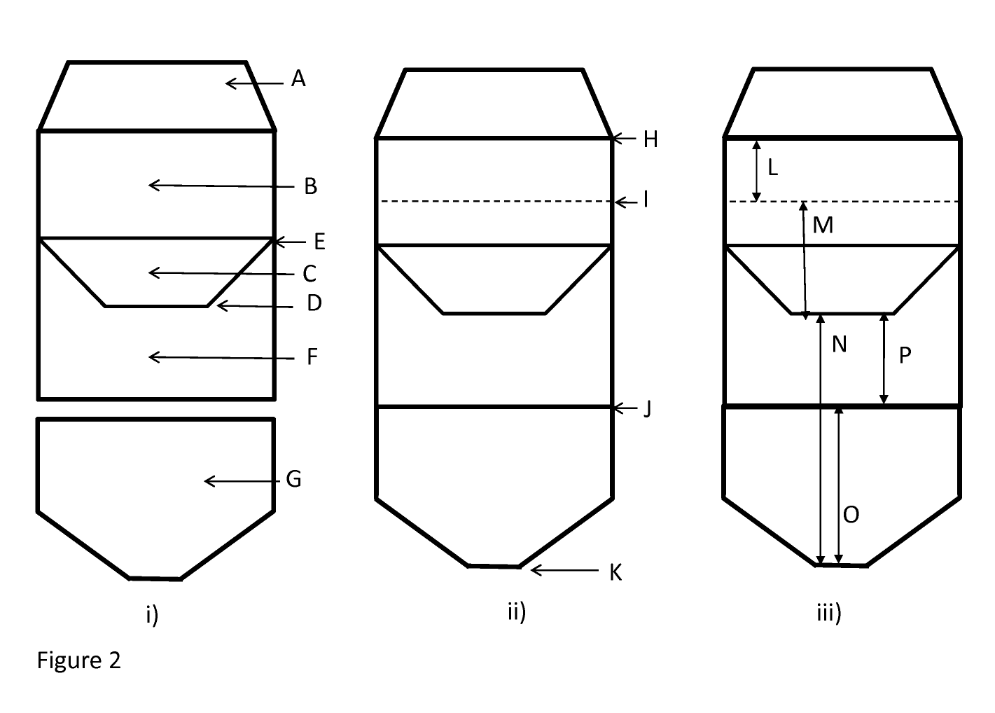

In one embodiment, the distance between the insert and the air-liquid interface (distance M in FIG. 2. iii) is minimized (distance M is less than distance N). Collection buffer is loaded into the lower chamber, and the Test Sample is loaded into the upper chamber. Separation is effected by gravity or centrifugation for a sufficient time for both 1) the non-buoyant cells to sediment, through the insert, into the bottom chamber and 2) the buoyant cells to accumulate at the air-liquid interface. This embodiment may be useful when accumulation of the buoyant particles at the air-liquid interface, and subsequent cessation of motion, is desired. For example, in the case of weak cell binding to microbubbles, detachment of the cell from the microbubble may occur during translation of the cell-microbubble complex. The apparatus as described here may avoid this situation by minimizing the distance that the buoyant particles must travel.

In one embodiment of the invention, the distance between the insert and the bottom of the bottom chamber (distance N in FIG. 2.iii) is maximized. The lower chamber is loaded with collection buffer, and the Test Sample is loaded into the upper chamber. Separation is effected by gravity or centrifugation for a sufficient time for the non-buoyant cells to sediment through the insert into the bottom chamber. Centrifugation is halted before cells reach the bottom of the bottom chamber and form a pellet. This embodiment may be useful, for example, in the case when pelleting or aggregation of the negative cells is not desired, for example to avoid cell damage or aggregation.

In one embodiment of the invention, the Test Sample is loaded into the upper chamber, and collection buffer is loaded into the lower chamber. This embodiment can be used in the case when the substance to be separated comprises cells. This embodiment is particularly useful in the case of negative selection, when a high purity is desired.

In one embodiment of the invention, the Test Sample is loaded into the lower chamber, and collection buffer is loaded into the upper chamber. This embodiment can be used in the case when the substance to be separated comprises cells or soluble analytes. This embodiment is particularly useful in the case of positive selection, when high purity is desired. This embodiment is particularly useful in the case of depletion.

In one embodiment, the top chamber of the apparatus comprises a cylindrical shape

In one embodiment, the bottom chamber of said apparatus comprises a cylindrical shape with a conical or rounded closed end at the bottom.

In some embodiments, the positive fraction is collected following the instant invention and subsequently discarded. This is the case, for example, when performing depletion.

In one embodiment of the invention, the total volume in the upper and lower chambers is between 1 and 5 mL.

In one embodiment of the invention, the total volume in the upper and lower chambers is between 5 and 50 mL.

In one embodiment of the invention, the total volume in the upper and lower chambers is between 0.2 and 1000 microliters.

In one embodiment of the invention, the ratio in diameter between the narrow (FIG. 3, measurement C) and wide (FIG. 3, measurement B) openings of the insert is between about 0.005 to about 0.5.

In one embodiment of the invention, the widest dimension of the upper chamber (FIG. 3, measurement A) is between about 1 mm and 50 cm.

In one embodiment of the invention, the connection between the upper chamber lid and upper chamber comprises a screw-type closure further comprising a flexible gasket and is airtight.

In one embodiment of the invention, the top chamber lid and bottom chamber are interchangable, so as to enable the same apparatus can be used for loading the test sample in either the bottom chamber or the top chamber.

In one embodiment of the invention, the total height of the apparatus (the sum of distances L, M, and N on FIG. 1.iii) is between about 1 cm and about 100 cm.

In one embodiment of the invention, the two-chamber apparatus is composed of a biocompatible material suitable for use with biological substances. In preferred embodiments, the two chamber apparatus is composed of one or more plastics or glass.

In one embodiment of the invention, incubation of the microbubbles and Test Sample occurs in the top chamber of the two-chamber apparatus. In another embodiment of the invention, incubation of the microbubbles and Test Sample occurs in the bottom chamber of the two chamber apparatus. In another embodiment of the invention, incubation of the microbubbles with the Test Sample occurs in a separate container, and the microbubble-cell dispersion is transferred to the two chamber apparatus prior to the separation procedure.

In one embodiment of the invention, the apparatus is pre-loaded with collection buffer before shipment to the end user. In some embodiments of the invention, the upper chamber is pre-loaded with collection buffer. In some embodiments of the invention, the lower chamber is pre-loaded with collection buffer. In some embodiments of the invention, both chambers are pre-loaded with collection buffer.

One embodiment of the invention comprises a kit for isolation of one or more targeted cell types from a single cell suspension, said kit further comprising one or more two-chamber apparatus' and microspheres.

In one embodiment of the invention, said microspheres further comprise the two-step “universal” microspheres of Example 5.

In one embodiment of the invention, said kit further comprises soluble second ligands for labeling of targeted cells by the end user.

In one embodiment the two chamber apparatus can be used for aseptic processing, for example in the context of cells isolated for downstream use as a therapeutic. In this case, the two chamber apparatus further comprises two or more luer-lock ports and plugs, to which sterile collection buffer, cells, and microbubbles can be added. After the separation procedure, the desired fraction (positive or negative) can be collected from the insert without breaking sterility by using the luer-lock ports and a sterile syringe. Cells collected can then be administered directly to the patient, or used for downstream processing prior to administration.

In one embodiment of the invention, the isolated cells comprise a diagnostic test, and said cells are utilized for downstream by PCR, DNA sequencing, culture, or functional assays. For example, isolated cells may comprise a bacterium, which may then be identified by culturing the isolated cells or performing genomic analysis.

### Collection of Positive and Negative Fractions

The two chamber apparatus provides a robust method to collect the positive and negative fractions following buoyancy-based separation.

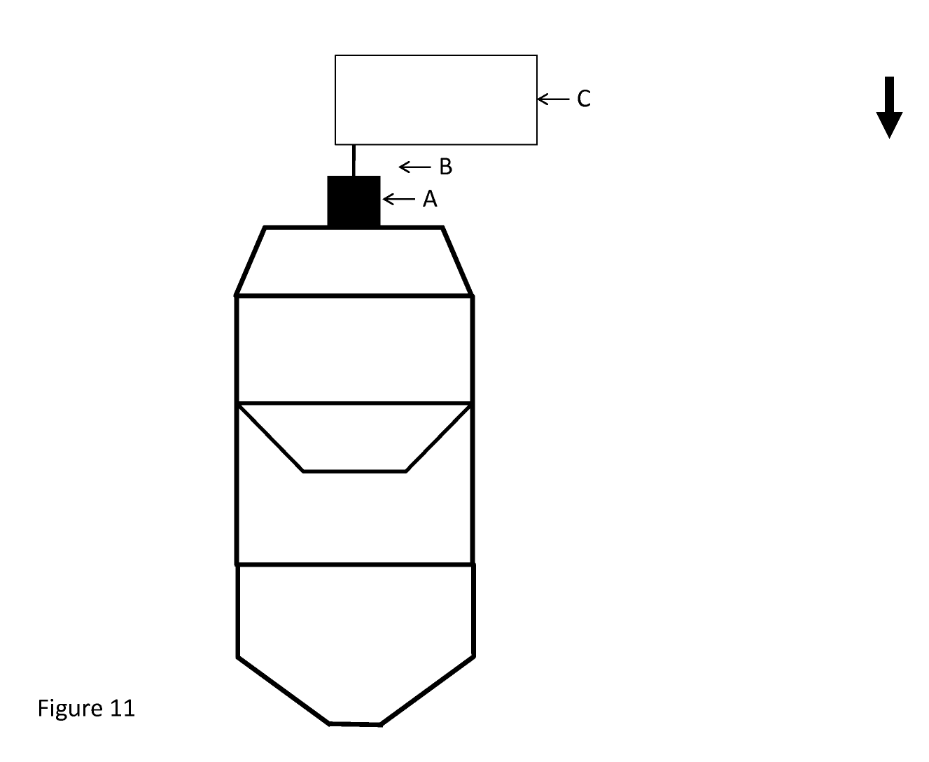

In one embodiment, the microbubbles are collapsed in the upper chamber of the two chamber apparatus. Such a scenario is depicted in FIG. 11. This may be accomplished, for example, by attaching a device (C) capable of generating increased pressure to the upper chamber by means of a port (A). Following collapse the positive fraction may be easily collected removing the top lid and decanting or collecting using a pipette. Exemplary pressure-generating devices include syringes, pumps and the like.

In another embodiment, the microbubbles and microbubble-cell complexes are removed from the top chamber and collapsed in a second container. Collection of the buoyant fraction from the upper chamber in this case may be accomplished by i) first re-dispersing the buoyant fraction by agitation, vortexing, or gentle shaking, and ii) decanting the contents of the upper chamber or collecting with a pipette. It should be noted that implementation of the insert as taught in the instant invention will prevent transfer of the sedimented (negative) population into the positive fraction during agitation.

### Methods of Loading the Two Chamber Apparatus

In one embodiment of the invention, the apparatus is fabricated so as to facilitate loading of the bottom chamber before loading of the top chamber.

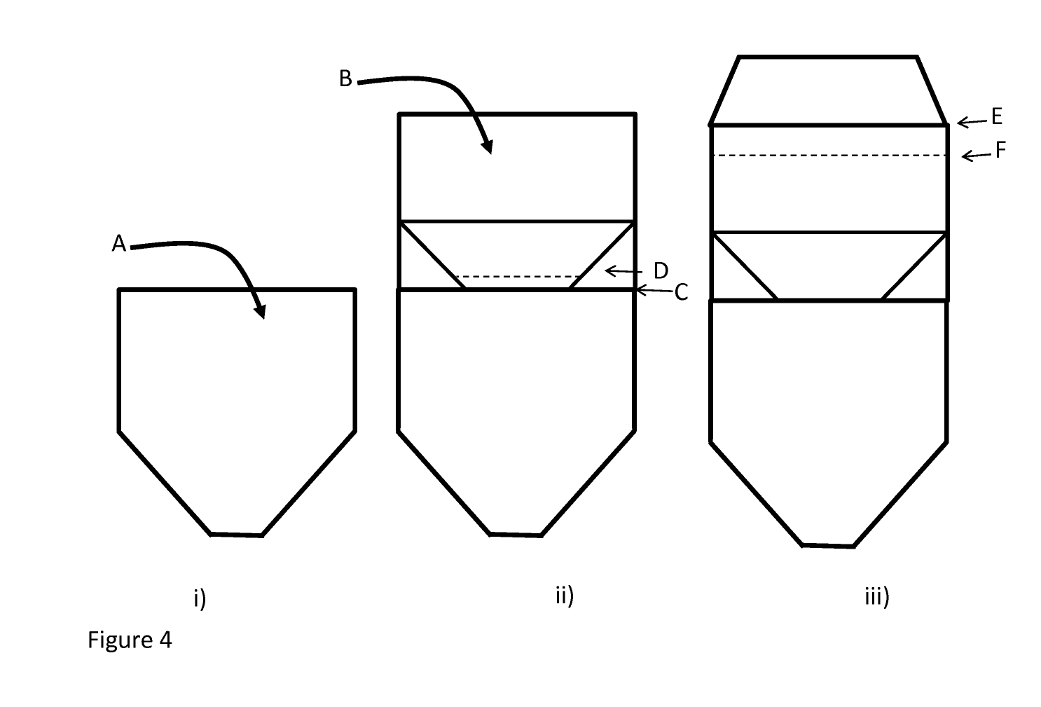

One embodiment, the apparatus is fabricated as shown in FIG. 4. The depth of the extender (distance P in FIG. 2) is desired to be significantly less than the depth of the bottom chamber (distance O in FIG. 2). The bottom chamber is first filled with collection buffer, using a pipette or other standard fluid-moving technique. The top chamber is then attached to the bottom chamber, forming an air-tight seal. In a preferred embodiment, a small amount of collection buffer may be present above the insert (dotted line in FIG. 4.ii). The Test Sample is then added to the upper chamber using a pipette or other fluid-moving technique. The top chamber lid is then attached, forming an air-tight seal (FIG. 4.iii, E). Separation may now be effected by centrifugation, and the positive and negative fractions collected as described above.

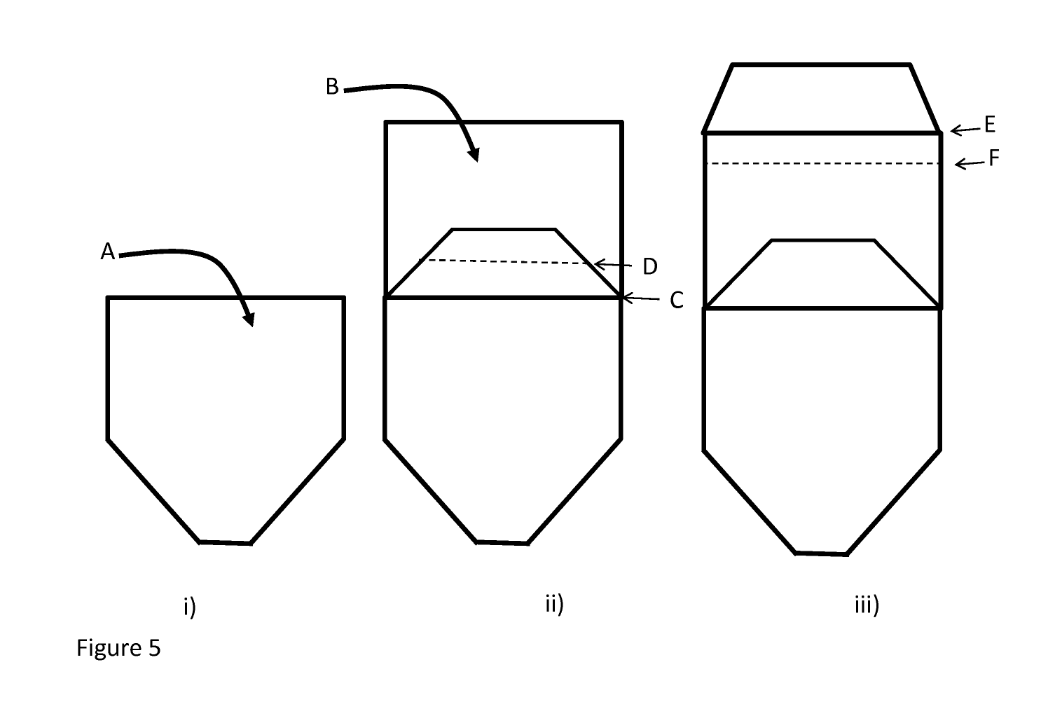

In another embodiment of the invention, the apparatus is prepared as shown in FIG. 5. The bottom chamber is first loaded with the Test Sample. The upper chamber, comprising the insert with the wide end facing the bottom chamber, is then attached to the bottom chamber as shown in FIG. 5. Collection buffer is then added to the upper chamber, and the upper lid attached. Separation may now be effected by centrifugation, and the positive and negative fractions collected as described above.

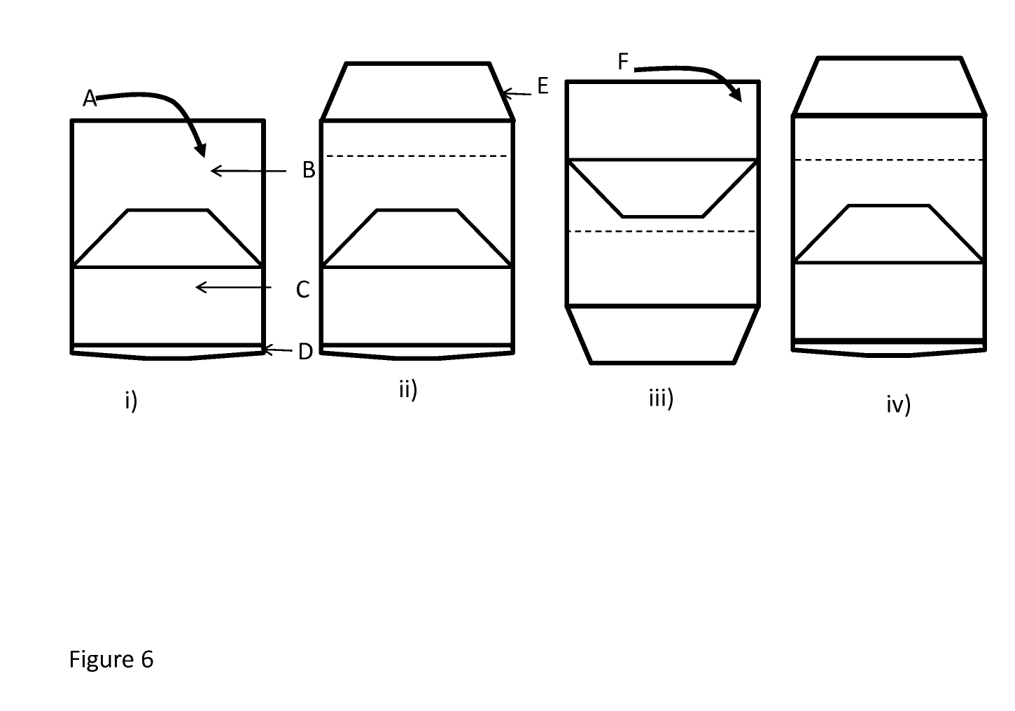

In one embodiment of the invention, the apparatus is fabricated so as to facilitate loading of the top chamber before loading the bottom chamber. For example, the apparatus is prepared as shown in FIG. 6. The depth of the bottom chamber (distance O in FIG. 2.iii) is minimized, such that distance O is less than distance P. Collection buffer is loaded to the upper chamber, and the top lid is secured. The apparatus is the inverted, and the bottom lid removed. The bottom chamber is then loaded with the Test Sample. It should be noted that, as described above, the dimensions of the upper chamber and the insert have been set such that none of the loaded fluid leaks through the insert into the upper chamber on loading. The bottom lid is then secured. The apparatus is then righted, and separation effected by centrifugation.

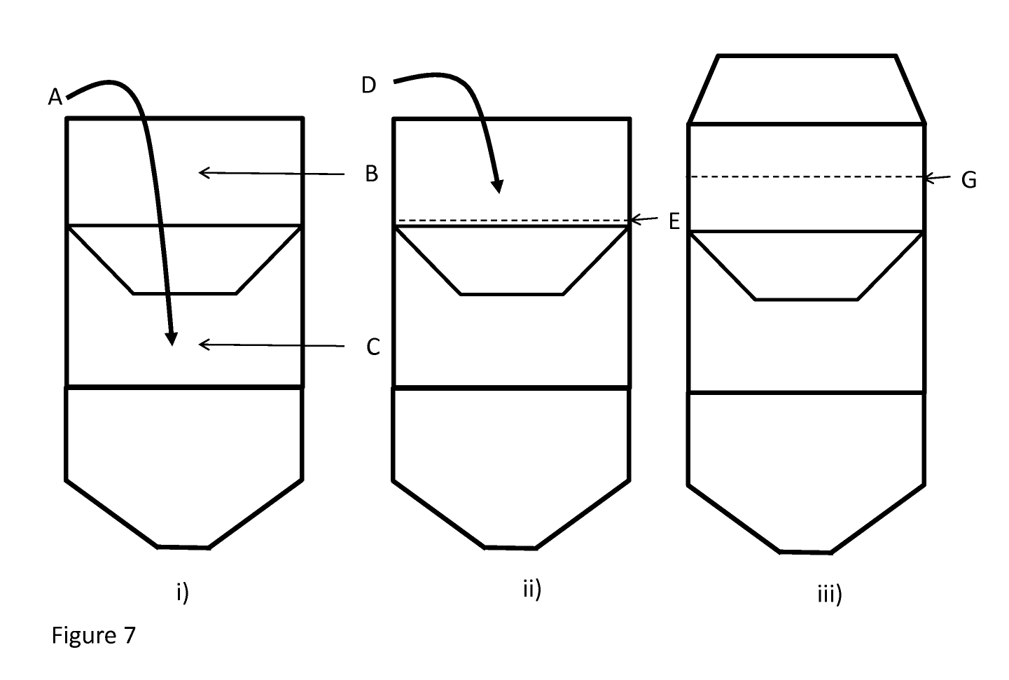

In one embodiment of the invention, detachment of the bottom chamber from the top chamber is not required for loading. For example, apparatus in which the Test Sample is loaded into the upper chamber may be prepared as described in FIG. 7. The collection buffer is first loaded, by placing the pipette tip or other fluid moving apparatus through the insert, into the bottom chamber. The Test Sample is then loaded onto the upper chamber. The top lid is then secured, and separation effected by centrifugation.

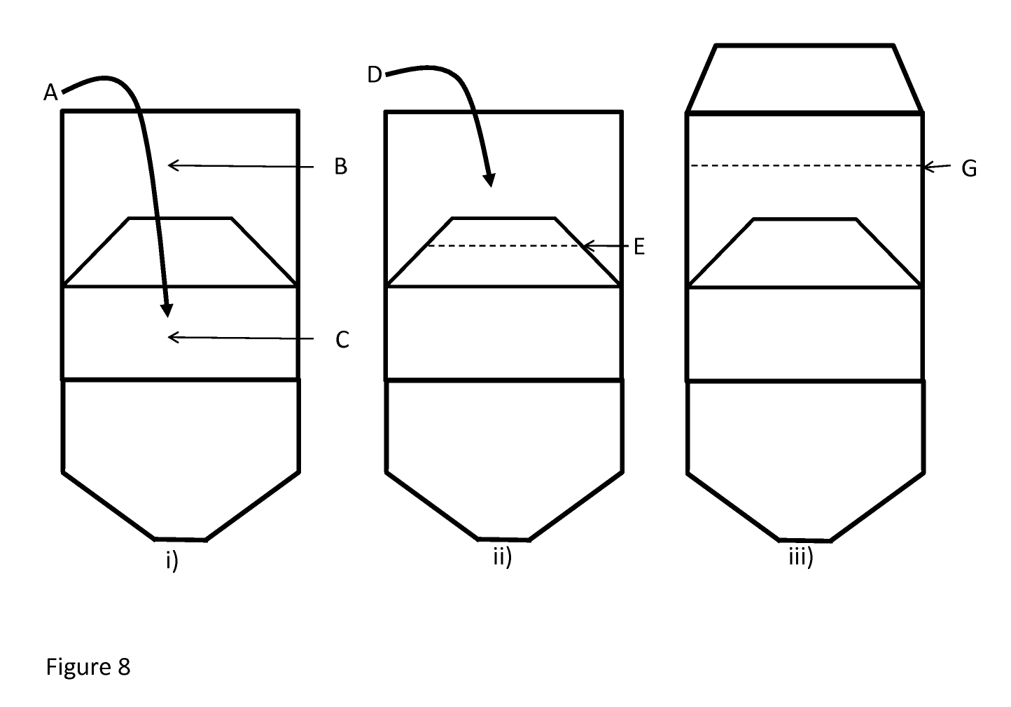

In another embodiment of the invention, an apparatus in which the Test Sample is loaded into the lower chamber may be prepared as described in FIG. 8. The Test Sample is first loaded, by placing the pipette tip or other fluid moving apparatus through the insert, into the bottom chamber. Collection buffer is then loaded onto the upper chamber. The top lid is then secured, and separation effected by centrifugation.

### Absence of Fluid Transfer Between Chambers

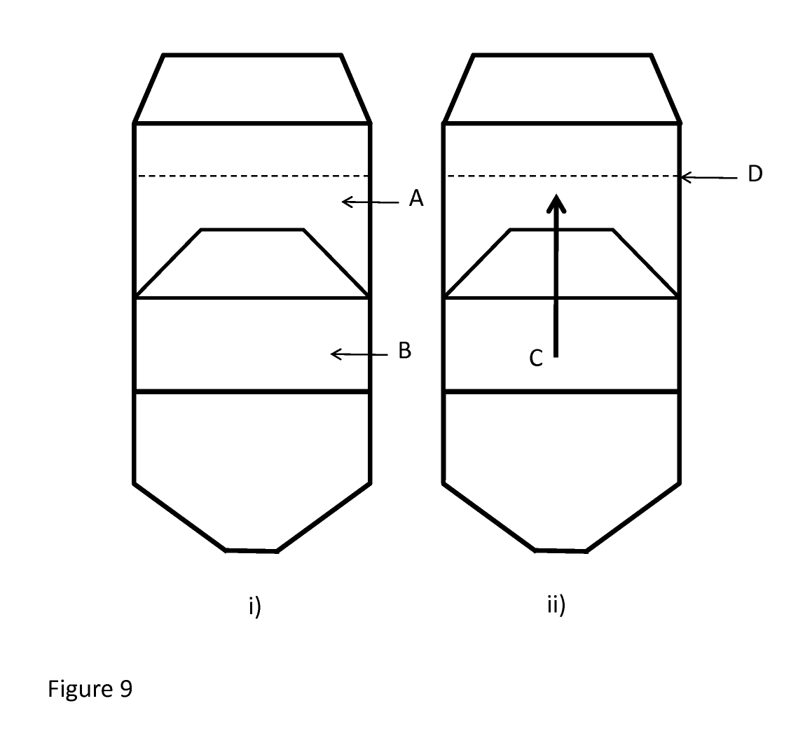

An important feature of the two chamber apparatus is that fluid is not transferred between chambers during the separation process. Rather, only particles are transferred. In the case of the Test Sample loaded into the lower chamber, separation is effected by the movement of the buoyant particles (comprising microbubbles and microbubble-cell complexes) from the lower chamber, through the insert, and into the upper chamber. The direction of buoyant particle motion is depicted by the arrow (C) in FIG. 9.ii. Pelleting of the negative (non-buoyant) cells on the bottom of the bottom chamber may occur, but is not necessary. Caking of the buoyant samples at the air-liquid interface (D in FIG. 9.ii) may occur, but is not necessary.

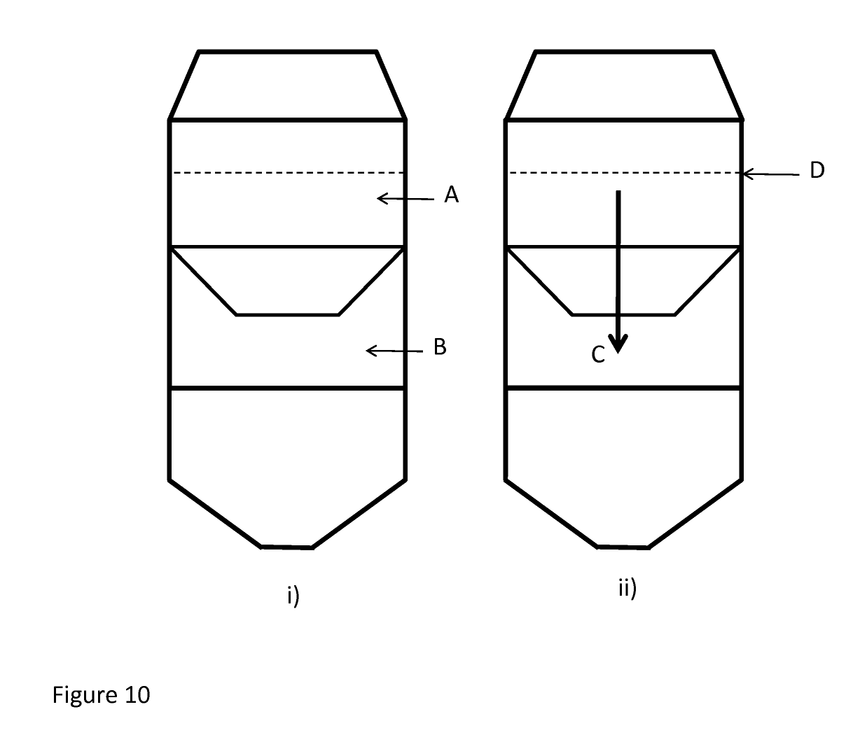

In the case of the Test Sample loaded into the upper chamber, separation is effected by movement of the non-buoyant particles (comprising non-microbubble bound cells) from the upper chamber, through the insert, and into the lower chamber by sedimentation. The direction of sedimenting particles is depicted by the arrow (C) in FIG. 10.ii. Pelleting of the negative (non-buoyant) cells on the bottom of the bottom chamber may occur, but is not necessary. Caking of the buoyant samples at the air-liquid interface (D in FIG. 10.ii) may occur, but is not necessary.

Another important feature of the two chamber apparatus is that the volume of each chamber is fixed throughout the separation procedure. This is in contrast to the apparatus described by Rongved (U.S. Publication No. US20070036722), which describes an apparatus of variable volume.

In one aspect, the invention provides a two-chamber system that enables a robust means of achieving buoyancy based cell separation under centrifugation. The system is constructed so that the microbubble-bound cells (positive fraction) and free cells (negative fraction) end up in chambers separated by a physical barrier at the end of the separation procedure. This overcomes a key problem with separation technologies using a buoyant medium, which is the difficulty in collecting a floating layer of microbubbles and microbubble-cell complexes.

In some embodiments, the present invention provides a two-chamber apparatus for use in separating target cells comprising a first top chamber with an opening at one end and further comprising a means for sealing said opening, a second bottom chamber with a closed end, wherein said first top chamber can be separated from said second bottom chamber.

### 4.3 Two-Stage Centrifugation

In some cases, detachment of the cells from the microbubble may occur during centrifugation. For example, in the case of cells in which the target is expressed in low density, or when using microbubbles comprising a ligand with very low density or very low affinity. In one embodiment of the instant invention, this may be avoided by utilizing a multi-stage centrifugation protocol. In this embodiment, a relatively low centrifugation speed, preferably between 1 and 200×G, is first applied to the sample for a sufficient time to effect concentration of substantially all of the microbubbles and attached target cells to the top of the upper chamber (or the gas-liquid interface). The centrifugation speed is selected so that the tension on the cell-microbubble bond, and commensurate cell-microbubble detachment, is minimized. Once microbubbles and attached cells reach the top of the upper chamber or gas-liquid interface, the cell-microbubble complex does not experience further movement, and the potential for cell-microbubble detachment is eliminated. At this point, a second, higher centrifugation speed may be implemented in order to sediment the negative, non-microbubble bound cells.

In a preferred embodiment, the first centrifugation step comprises two minutes at 50×G and the second centrifugation step comprises five minutes at 500×G.

It will be apparent to one skilled in the art that the two-stage centrifugation protocol described here may be readily implemented with the two-chamber device or with virtually any other container (including centrifuge tubes, conical tubes, spin columns, and the like) suitable for centrifugation of cells.

### 4.4. Isolation of Soluble Analytes

Microbubbles and methods taught in the instant invention are also applicable for isolation of soluble analytes in addition to cells. In this context the microbubbles comprise a targeting ligand specific for the soluble analyte. Selection of targeting ligands with high affinity is of key importance in this application, as the formation of multiple microbubble-analyte bonds may not be feasible.

In a preferred embodiment, microbubbles are used for depletion of the soluble analyte. For example, microbubbles comprising an antibody against TNF-alpha may be used to clear a liquid sample comprising an aqueous buffer and suspended cells.

In some cases, the soluble analyte bound to the microbubble may be utilized in downstream analysis. For example, microbubbles comprising a ligand specific for viral particles may be used to clear a liquid sample of virus. The microbubbles may be collected using the positive selection method taught in the instant invention, and the adherent viral particles may be analyzed. For example, virus-bearing microbubbles may be stained with one or more fluorescently-labeled antibody specific for viral components of interest, and the concentration or composition of isolated virus analyzed by flow cytometry. For example, microbubbles and attached virus may be denatured and nucleic acid from the virus extracted for molecular analysis.

### 4.5. One-Step and Multi-Step Separation

In some circumstances, it is desirable to prepare microbubbles bearing a ligand that binds to the cells of interest for buoyancy-based separation. Such microbubbles enable one-step cell separation, whereby the microbubbles are incubated with the previously unmodified mixed cell population, and the targeted cells are collected by the buoyancy-based procedure described in the instant invention. For example, a CD34-binding peptide may be used as a ligand on the microbubble surface, and used for positive selection of CD34 stem cells from human leukapheresis material following the method described above. This method provides for a relatively rapid and robust method of cell separation. From a commercial perspective, the advantages of the one-step system are that it enables optimization of the microbubble properties (ligand density, diameter, etc) and procedure (centrifugation time and duration) for each individual cell type.

Exemplary ligands for one-step separation include ligands that recognize cell surface proteins such as antibodies, ligands that recognize viral-peptides, ligands that recognize antigen-presentation complexes, multimeric complexes comprising MHC I or MHC II and antigen peptide, MHC I or MHC II tetramers or dendrimers, ligands that recognize exogenously introduced cell surface markers.

Kits comprising microbubbles bearing ligands specific for the cell type of interest and a protocol detailing the optimized separation conditions are envisioned. In a preferred embodiment, said kit also comprises a two-chamber insert suitable for use in the specified application.

In some circumstances, it is desirable to prepare a microbubble bearing a ligand that binds to a second ligand, in which said second ligand binds to the cells of interest. Such microbubbles enable two-step cell separation, whereby 1) the cells are incubated with the second ligand and 2) ligand-labeled cells are then labeled with the microbubbles, and the targeted cells are collected by the buoyancy-based procedure described in the instant invention. The microbubble ligand may bind to a common region found on multiple variants of a second ligand, enabling a single microbubble-ligand formulation to be useful a wide variety of applications (each of which comprise a unique second ligand). For example, the microbubble ligand may bind to biotin, allowing separation of cells that are stained with a biotinylated antibody. The two-step system provides for a “universal” microbubble, able to be used with a wide diversity of second ligands.

Another advantage of the two-step system pertains to the simplicity of a negative selection kit, whereby a diversity of cells can be first labeled using a cocktail of ligands specific for the various types of cells to be targeted, then incubated with a single microbubble formulation, wherein the ligand on the microbubble recognizes a conserved region on the ligand used to label the cells.

Exemplary first ligands are avidin, streptavidin and other biotin-binding proteins, biotin-binding peptides, anti-biotin antibodies, ligands that recognize conserved domains of antibodies, anti-Fc domain antibodies, ligands that recognize fluorochromes, ligands that recognize annexin V, ligands that recognize lanthanides or to other stable metals, ligands that recognize affinity tags,

Exemplary second ligands include antibodies, ligands conjugated to biotin, Fc-bearing proteins, ligands conjugated to phycoerythrin (PE), FITC, APC, or to other fluorochromes, ligands conjugated to lanthanides or other stable metals, annexin v, ligands that recognize cell surface markers of apoptosis including phosphatidylserine, ligands bearing affinity tags including poly-His tags, FLAG tag, strep-tag, or Myc-tag.

It will be apparent to one skilled in the art that this strategy can be extended to more than one ligand on the surface of the microbubble. For example, microbubbles may be prepared with antibodies against FITC and antibodies against PE. Targeted cells may be stained with a diversity of antibodies, comprising either FITC or PE. The single double-ligand microbubble may be then used to isolate cells stained with either 1) FITC or 2) PE, or 3) both FITC and PE. This arrangement may be advantageous, for example, in increasing the yield for rare targeted cells (e.g., <1% of the starting population), or in the case where a single ligand is not able to adequately identify the targeted cell.

Kits comprising microbubbles bearing an exemplary first ligand are contemplated. In a preferred embodiment, said kit also comprises a two-chamber insert. In some embodiments, one or more second ligands are included in said kit. In some embodiments, second ligands are procured independently of the kit.

In some circumstances, it is desirable to prepare a microbubble bearing a ligand that binds to a second ligand, in which said second ligand binds to a third ligand, in which said third ligand binds to the cells of interest. Such microbubbles enable multi-step cell separation, whereby 1) the cells are incubated with the third ligand and 2) the cells are incubated with the second ligand, and 3) ligand-labeled cells are then labeled with the microbubbles, and the targeted cells are collected by the buoyancy-based procedure described in the instant invention. This multi-step separation strategy may be useful for isolating cells in which the target of interest is expressed in low copy number, or for which ligands that bind with high affinity are not available.

For example, cells may be stained first with an antibody raised in rat, subsequently stained with an antibody raised in mouse (i.e., a mouse-anti-rat antibody), and subsequently incubated microbubbles bearing an anti-mouse antibody.

Amplification is desirable in the multi-step separation process described here. For example, it is desirable that multiple molecules of the third ligand bind to each molecule of the second ligand. For example, it is desirable that multiple molecules of the first ligand bind to each molecule of the third ligand.

For example, cells are incubated with a second ligand bearing multiple (2-10) biotin molecules. The cells are subsequently incubated with a third ligand comprising an anti-biotin antibody conjugated to FITC. Each anti-biotin antibody is able to bind the biotin at multiple sites on the second ligand, due to the presence of the multiple copies of biotin on said second ligand. A microbubble bearing a first ligand comprising an antibody against FITC is then used to isolate the labeled cells.

In a preferred embodiment, the multi-step procedure described above may comprise between 3-5 steps.

In the case of a two-step or multi-step isolation system, uses of second ligands containing a moiety detectible by a conventional assay are desirable. For example, second ligands comprising antibodies conjugated to fluorochromes are desirable, as they enable identification of the targeted cells by fluorescence-based methods such as flow cytometry and fluorescence microscopy. This eliminates the problem of the first antibody occupying the target site and preventing staining for subsequent identification, as frequently occurs in the case for one-step cell separation.

It should be apparent to one skilled in the art that the one-, two-, and multi-step isolation procedures described above are suitable for use in both positive and negative selection applications.

### 4.6. Sequential Separation

Separation procedures occurring multiple times in sequence are also contemplated in this invention. The collapsible microbubble makes multiple sequential separation feasible, in that desired cells may be selected using a first microbubble composition, then collapsing said first microbubbles leaving a subset of the collected cells which are then further selected by combining the subset of cells with a second microbubble composition. This may be particularly useful for isolating complex cells defined by the expression of more than one cell surface marker and found in a mixed cell population in which unwanted cells express one or more of said cell surface markers.

For example, regulatory T-cells are defined by the expression of both CD4 and CD25 (CD4+/CD25+). Each of these markers (CD25 and CD4) are expressed separately on different cell types found in mouse spleen homogenate

Accordingly, another embodiment of the invention provides for sequential multi-process method of separating target cells from a mixed cell population in a liquid sample, the method comprising the steps of: i. mixing the cells with an aqueous solution containing more than one ligands, each labeled with a distinct marker group, ii. mixing the labeled cells with a first buoyant microbubble composition, iii. incubating the liquid sample at a temperature between 4° C. and 37° C. for a sufficient time to allow the target cells and the first microbubbles to form cell-microbubble complexes, iv. applying sufficient gravitational force to the liquid sample containing the cell-microbubble complexes for a sufficient period of time to effect separation of the buoyant cells, vi. Removing the microbubble by collapse, thereby liberating the target cell from the microbubble-cell complex, vii. collecting the target cells, viii. mixing the collected cells with a second buoyant microbubble composition, wherein said microbubble composition comprises a ligand specific for a different marker group, and, ix. repeating steps iii-viii one or more times until the positive fraction comprises only cells bearing all of the desired targets.

For example, regulatory T-cells found within mouse spleen homogenate may be first stained with a FITC-conjugated anti-CD4 antibody and a PE-stained CD25 antibody. Stained splenocytes may then be incubated with a first microbubble formulation comprising an anti-FITC antibody, and cells bearing the CD4-FITC antibody isolated by positive selection. The microbubbles may then be removed by collapse, and the resulting cells incubated with a second microbubble formulation comprising an anti-PE microbubble. The cells bearing the CD25-PE antibody are then isolated by positive selection, providing a positive fraction enriched in cells that are positive for both CD24 and CD4.

In one embodiment, the sequential separation procedure is performed between 2 to 5 times.

In one embodiment, the sequential separation procedure comprises both positive and negative selection steps.

### 4.7 Negative Selection

Desired cells can be isolated from a complex mixture by negative selection in the context of the present invention. This can be achieved using a negative selection separation scheme. Soluble ligands specific for cell surface markers found only on the un-desired cell(s) are added to the mixed cell population, and incubated for sufficient time for said ligands to bind to the targeted cells. Said ligands further comprise a marker group, such as biotin, phycoerythrin, or colloidal gold. The cell suspension may be washed, for example by centrifugation, to remove any residual ligand not bound to cells. The cell suspension is then incubated with a buoyant microbubble composition the buoyant fraction isolated from the non-buoyant fraction by centrifugation. The buoyant fraction, comprising the microbubbles and adherent targeted cells, is discarded, and the sedimented cells are retained.

As such, another aspect of the invention is a method of separating a complex mixture of cells in an aqueous environment comprising negative selection of cells. The method comprises the steps of i. mixing the cells with an aqueous solution containing one or more ligands labeled with a marker group, ii. mixing the solution from step (i) with a buoyant microbubble composition; iii. incubating the liquid sample at a temperature between 4° C. and 37° C. for a sufficient time to allow the target cells and the microbubbles to form cell-microbubble complexes, iv. applying sufficient gravitational force to the liquid sample containing the cell-microbubble complexes in said two-chamber apparatus for a sufficient period of time to cause the cell-microbubble complex to become enriched in the upper chamber of said two-chamber apparatus, and the remaining cell population to become enriched in the bottom chamber of said two-chamber apparatus, v. separating said bottom chamber of the two-chamber apparatus from said top chamber of apparatus of the two-chamber apparatus wherein the top chamber contains the cell-microbubble complexes and the bottom chamber contains the free cells, vi. collecting the free cells.

### V. Examples

The following examples are provided by way of illustration only and not by way of limitation. Those of skill will readily recognize a variety of noncritical parameters that could be changed or modified to yield essentially similar results.

### Example 1

### Synthesis of Uncharged Lipid Microbubbles Containing Decafluorobutane Gas

Microbubbles consisting of a decafluorocarbon gas core encapsulated by a two-surfactant shell were prepared as follows. 100 mg of the lipid disteroylphosphatidylcholine (Avanti) and 50 mg of the surfactant PEG-40 stearate (Sigma) were solubilized by low-power sonication of 20 minutes at 9 W (CP-505: Cole-Parmer) in 0.9% injection grade NaCl (normal saline; Baxter). The mixture was heated to 70° C., and microbubbles formed by high-power sonication (30 s at 40 W) while sparging decafluorobutane gas (Fluoromed). This procedure results in the formation of a polydisperse, right-skewed dispersion of lipid-stabilized microbubbles of decafluorobutane, at a concentration of 2-4E9 per mL, number-weighted mean diameter of 2 μm, and 95% between 1-4 μm. The resulting microbubble dispersion was then allowed to cool to room temperature. Shell forming materials not incorporated into microbubbles were removed by centrifuging the dispersion for 10 minutes at 1000×G, 15° C. (Allegra 6R bucket centrifuge: Beckman-Coulter) in a 100 mL sealed glass vial with a decafluorobutane gas headspace and collecting the infranatant with a thin needle. Microbubbles were then re-suspended at a concentration of 4E9 per mL in a buffer consisting of 300 g/L glycerin, 300 g/L propylene glycol in normal saline, pH 5-6.5 (saline/glycerin/propylene glycol buffer). Chromatographic analysis of the microbubbles revealed that the DSPC composed approximately 95% of the shell, and PEG-stearate the remainder.

It should be apparent that substitution of the surfactant (PEG-40 stearate) above with other amphipathic substances is within the scope of the present invention, provided that said substances have a higher water solubility than the lipid Second surfactants which consist of a hydrophobic anchoring component (such as a fatty acid) grafted to a hydrophilic polymer (such as polyethylene glycol) are especially preferred. Examples of surfactants include polyethyleneglycol (PEG) esters of fatty acids, PEG-linked ceramides, PEG-linked phospholipids, phospholipid-polyglycerine derivatives, PEG-linked cholesterol, fatty acid esters, and fatty alcohols. The linked polymer may exist as a linear, branched, or comb configuration.

### Example 2

### Synthesis of Microbubbles with Reactive Groups Suitable for Ligand Conjugation on the Surface

Microbubbles suitable for conjugation of a ligand were prepared by incorporating into the lipid/emulsifier blend a conjugation residue immobilized on a hydrophobic anchor. Microbubbles bearing the protected sulfhydryl reactive group 2-pyridyl disulfide were prepared as follows. One hundred mg of disteroylphosphatidylcholine, fifty mg polyoxyethylene 40 stearate, and 1.25 mg of PDP-PEG(2000)-disteroylphosphatidylethanolamine (DSPE-PEG(2k)-PDP; Avanti) was added to 20 mL of sterile normal saline and sonicated to clarity using a probe-type sonicator. Microbubbles were formed and washed as in Example 1. This procedure results in the formation of a polydisperse dispersion of lipid-stabilized microbubbles of decafluorobutane, at a concentration of 2-4E9 per mL, number-weighted mean diameter of 2 μm, and 95% between 1-4 μm. Microbubbles were re-suspended at a surface area concentration of 2E11 μm²/mL in Dulbecco's phosphate buffered solution (DPBS) containing 300 mg/mL of glycerin and 300 mg/mL of propylene glycol, pH 7.4 (DPBS/glycerin/propylene glycol). Microbubbles were stored in sealed glass vials under a headspace of decafluorobutane gas until ready for use.

In a separate experiment, similar microbubbles were prepared in which the PEG group of the emulsifier was anchored to a lipid. Fifty mg of DSPE-PEG(1k), 100 mg of DSPC, and 5 mg of DSPE-PEG(2k)-PDP was added to 50 mL of normal saline and sonicated to clarity. Microbubbles were prepared as described above. This procedure resulted in the formation of a polydisperse dispersion of lipid-stabilized microbubbles of decafluorobutane, at a concentration of 2-4E9 per mL, number-weighted mean diameter of 1.4 μm and >90% between 1-2 μm. These microbubbles were washed and stored as described above.

In the examples above, the reactive group (PDP) was immobilized on the distal tip of a PEG grafted to a phospholipid anchor. In the case when the surfactant also uses an extensible hydrophilic component, it is desirable that the reactive group be immobilized on a longer extensible hydrophilic component. For example, when the emulsifier is DSPE-PEG(1k), the use of a longer PEG (DSPE-PEG(2k) to anchor the reactive group is preferred.

Incorporation of the PDP residue enables conjugation of a targeting ligand to the microbubble surface via sulfhydryl-directed conjugation chemistry. Various other ligand conjugation chemistries can be readily used by substituting for the DSPE-PEG(2000)-PDP component. For example, microbubbles bearing biotin (suitable for binding a biotinylated ligand via an avidin-based linker) are be prepared by the inclusion of 5 mg/mL biotin-PEG(2000)-DSPE. Alternatively, ligands can be immobilized via thioether linkage by incorporating 5 mg/mL of maleimide-PEG(2000)-DSPE. Other reactive groups suitable for ligand conjugation include amino, hydroxyl, carboxyl, carbonyl, n-hydroxysuccinimide, carbohydrates, epoxy, cyanur, 2-aminoalcohols, 2-aminothiols, azide, alkyne, alkoxyamine, aldehydes, guanidinyl groups, imidazolyl groups, and phenolic groups.

It will be obvious to one skilled in the art that the density of the reactive group within the microbubble shell can be modulated by the mass fraction of the reactive group added during the synthesis step.

### Example 3

### Conjugation of an Antibody to the Surface of Functionalized Microbubbles

A monoclonal antibody specific for CD8 (a cell receptor found on a subset of T-cells) was chosen as a ligand to enable recognition of specific cell types in this experiment. A rat anti-mouse antibody specific for CD8 (Clone 53-6.7; eBioscience) was concentrated to >2 mg/mL in 0.1M sodium acetate buffer (pH 5.5). Carbohydrate residues on the antibody were oxidized by incubation with 10 mM sodium periodate for 30 min at room temperature. The antibody was exchanged into fresh acetate buffer and incubated with the heterobifunctional crosslinker PDPH (pyridyldithiol-and-hydrazide) (5 mM) and 0.9% aniline for 1 hour at room temperature. The antibody was then purified by gel filtration into DPBS with 10 mM EDTA, pH 7.4. This procedure resulted in derivitization of the antibody with a protected thiol group preferentially bound to the Fe region. The derivatized antibody was stored at high concentration (>2 mg/mL) at 4 deg C until ready for use.

Microbubbles prepared with a PDP residue were prepared as described in Example 5. The microbubbles were incubated with 1 mM tris(2-carboxyethyl)phosphine-based reducing agent (TCEP; Pierce) to convert the stable PDP residue to the reactive sulfhydryl form. Reducing agent and reduction bi-product was removed by washing the microbubbles three times at 15 deg C in DPBS/glycerin/propylene glycol buffer. Microbubbles were concentrated to 2E11 μm²/mL in a final volume of 1.0 mL. 5.0 mg of the PDPH-conjugated antibody was added to the concentrated microbubble dispersion, and allowed to react for 16 hours in a sealed glass vial under a perfluorocarbon headspace with gentle end-to-end rotation at 4 deg C. Unreacted antibody was removed by centrifugation of the microbubbles under C₄F₁₀ gas at 1000×G for 10 minutes. The MBs were resuspended with 1 mL of sodium borate buffer, pH 8.5 with 50 mM iodoacetamide. The MBs were allowed to react for 1 hour in a glass vial under perfluorocarbon headspace with gentle end-to end rotation at room temperature. Unreacted iodoacetamide and antibody was removed by three washes in glycerin/propylene glycol buffer. Microbubbles were re-concentrated to 2E9 per mL and stored in a 3.0 mL glass vial with a headspace of decafluorobutane gas.

Successful conjugation of the antibody to the microbubble surface was verified by flow cytometry and immunoassay. Five microliters of the microbubble dispersion was incubated with a FITC-conjugated anti-rat IgG for 20 minutes at room temperature. Microbubbles were analyzed in the flow cytometer (Guava; EMD Millipore) for the presence of FITC (green).

Microbubbles were diluted to 2E9 MB/mL. Microbubbles were diluted 1:1, then blotted into a fresh nitrocellulose membrane. The nitrocellulose was blocked by incubating with a solution of dry milk powder, and then incubated with his-tagged recombinant mouse CD8 (rmCD8-His) protein. An HRP-conjugate anti-His antibody was then incubated with the nitrocellulose, and unreacted antibody removed by washing the membrane. Binding activity was accessed by the formation of 3,3′,5,5′-tetramethylbenzidine diamine when incubated with 3,3′,5,5′-tetramethylbenzidine solution. Blots in which the antibody-conjugated microbubbles were used developed a brown color, while blots in which naked microbubbles, or microbubbles bearing an isotype control antibody, did not develop color.

It is to be understood that this technique is not limited to the particular antibody described above, and this method can be used by one skilled in the art to conjugate essentially any antibody. Antibodies against human CD8, mouse Ly6G, mouse MAdCAM-1, mouse CD4 and mouse CD19 were conjugated to the microbubble using the method presented above, with similar results (Table 5).

TABLE 5
Summary results of directional antibody conjugation experiments.

| | Mean Diameter | Antibody | |
|---|---|---|---|
| | of Microbubble | Binding | |
| Antigen | (μ(m) | (% Positive) | Immunoblot Result |
| Mouse CD8 | 2.417 | 99.1 | Positive reaction |
| Mouse CD19 | 2.406 | 98.7 | Positive reaction |
| Human CD8 | 2.039 | 40.6 | Positive reaction |
| Mouse Ly6G | 2.117 | 82.8 | Positive reaction |
| Mouse CD4 | 2.018 | 99.0 | Positive reaction |
| Mouse | 2.184 | 98.0 | Positive reaction |
| MadCAM1 | | | |

Moreover, similar bioconjugation techniques can be used to label the microbubble with diverse other target-binding ligands, including proteins, peptides, aptamers, nucleic acids, single chain antibodies and other immunoglobulin fragments.

### Example 4

### Preparation of Microbubbles with Varying Ligand Density on the Microbubble Surface

It is possible to modulate the density of ligand bound to the surface of the microbubble by adjusting the relative concentration of antibody and reactive microbubble groups during the incubation step. Microbubbles were prepared using the method described above in example 3. To vary the ligand density, microbubbles were concentrated to 2E11 μm²/mL in a final volume of 1.0 mL and were incubated with 0.05-5.0 mg of the PDPH-conjugated antibody, and allowed to react for 16 hours in a glass vial under a perfluorocarbon headspace with gentle end-to-end rotation at 4 deg C. Microbubbles were blocked with iodoacetamide and washed following example 3.

TABLE 6
Summary of results of varying ligand
density on the microbubble surface

| Anti-Mouse CD19 | | Anti-Mouse MadCAM1 | |
|---|---|---|---|
| Mg of mouse | Antibody Binding | Mg of mouse | Antibody Binding |
| CD19 | (Percent Positive) | MadCAM1 | (Percent Positive) |
| 1.0 | 9.2 | 0.05 | 0.3 |
| 2.0 | 37.2 | 0.5 | 76.7 |
| 2.5 | 96.7 | 2.5 | 98.5 |
| 5.0 | 98.0 | 5 | 98.3 |

In a separate experiment, the density of an anti-PE antibody was controlled by varying the density of the anchor molecule. Microbubbles were prepared as in examples 3-5, with a density of DSPE-PEG(2k)-PDP of between 0.01 and 1.0% by moles. Microbubbles were incubated with excess antibody (two antibodies were investigated: clone DLF or clone APC-6A2) as in the previous example 3. Antibody density was assessed by ELISA, and was found to vary approximately linearly with anchor density over the range assessed here.

### Example 5

### Preparation of a “Universal” Microbubble for Two-Step Cell Separation

A “universal” microbubble for cell separation can be prepared by incorporating a ligand able to recognize the target cell through a second targeting ligand bearing the appropriate marker group. For example, targeted cells may first be labeled with a biotinylated antibody. A streptavidin-coated microbubble may then be used to isolate the targeted cells by virtue of the biotin-streptavidin binding interaction. This provides for a two-step cell separation system, which is advantageous in that one microbubble formulation can be used with a wide variety of cell-binding ligands.

Streptavidin-coated microbubbles were prepared as follows. Twenty-five mg of streptavidin was dissolved in DPBS at a concentration of 5 mg/mL, and reacted with 3.26 mg of the heterobifunctional crosslinker N-succinimidyl 3-2(2-pyridyldithio)-propionate) (SPDP; Pierce) for 30 minutes at room temperature. Unreacted crosslinker was removed by gel filtration. SPDP-streptavidin was incubated with 25 mM dithiothreitol (DTT) for 30 minutes to expose a reactive sulfhydryl group on the crosslinker, and purified by gel filtration. Microbubbles bearing a PDP residue were prepared as described in Example 2 and diluted to a surface concentration of 2E11 μm²/mL in DPBS/glycerin/propylene glycol buffer. Twenty-five mg of sulfhydryl antibody was added to 12.54 mL of microbubbles, and incubated for 16 hours at 4 deg C with gentle end-to-end agitation. Unreacted streptavidin was removed by three rounds of centrifugal washing, and microbubbles were re-suspended in DPBS/glycerol/propylene glycol buffer at a concentration of 2E9 per mL in a glass vial under a headspace of decafluorobutane gas.

The density of streptavidin on the microbubble surface, and the functionality thereof, was assayed by flow cytometry and ELISA, as follows. Microbubbles were diluted to 500E6 microbubbles/mL. 5 μL of the microbubble was incubated with 12 μL of 10 μg/mL a FITC and biotin labeled IgG for 20 minutes at room temperature. The mixture was vortexed every 5-8 minutes during incubation. Microbubbles were analyzed in a flow cytometer (Guava; EMD Millipore) and the intensity in the green channel quantified. Over 95% of microbubbles prepared by this method exhibited a positive FITC signal relative to control microbubbles bearing only the IAM quenching group, demonstrating successful antibody conjugation.

It will be clear to one skilled in the art that various other receptor/ligand pairs besides biotin/streptavidin are suitable for use in the context of a universal two-step cell separation microbubble. Molecules that are routinely conjugated to antibodies and other specific ligands, such as fluorophores, metals, radioisotopes, haptans, polyhistidine tags are especially useful; substances that recognize said molecules can be placed on the microbubble using the conjugation schemes described here. Of particular utility are fluorophore and anti-fluorophore antibody pairs, wherein the fluorophore is conjugated to the cell-recognizing antibody and the anti-fluorophore antibody is conjugated to the microbubble.

### Example 6

### Preparation of Monodisperse Microbubbles

The size distribution of the microbubbles prepared in the preceding examples can be tuned to yield an essentially monodisperse population of a desired diameter. This is desirable in order to match the magnitude of the buoyant force to the size and density of the target molecule on targeted cell, and to control the degree of interaction between the microbubbles and the cells. It was found that the method in this example was exceptionally efficient, and resulted in a high yield of stable monodisperse microbubbles, when used with microbubbles prepared with an emulsifier consisting of at least 100 ethyleneglycol units attached to a hydrophobic anchor. Microbubbles were prepared by first solubilizing 100 mg of DSPC and 140 mg of disteroylphosphatidylethanolamine-PEG-5000 in 50 mL of hot saline by low-power sonication. Microbubbles were then formed by high-power sonication at the gas-liquid interface while sparging decafluorobutane gas. Microbubbles were then centrifuged under a headspace of C₄F₁₀ gas for 10 minutes at 1000×G, and 45 mL of infranatant was removed and discarded. 45 mL of saline/glycerin/propylene glycol buffer was then added. The dispersion was centrifuged for 2 minutes at 200×G under a headspace of decafluorobutane gas, and 45 mL of infranatant was collected and stored in a glass vial under a decafluorobutane gas headspace. Electrozone sensing revealed the collected microbubbles to have a mean diameter of 1.6 m and <0.01% greater than 3.0 μm.

It will be obvious to one skilled in the art that monodisperse microbubbles prepared by this method can be readily conjugated to targeting ligands using the methods described in Examples 3-5.

### Example 7

### Preparation of Small Microbubbles with Symmetric Size Distribution

Microbubbles exhibiting a symmetric size distribution, with a number-weighted mean diameter of 1.4 m and <20%/above 2 m, were prepared as follows. PDP-bearing microbubbles were synthesized as described in Example 2. Microbubbles were diluted in 50 mL of normal saline/glycerin/propylene glycol buffer. Microbubbles were centrifuged in a 100 mL glass vial containing a headspace of 50 mL of decafluorobutane gas for 2 minutes at 200×G. This resulted in the formation of a “cake” containing foam and very large microbubbles at the top of the vial, with a clear demarcation between the cake and infranatant. Forty five mL of infranatant was collected by inserting a sterile 19G needle through the vial septum and slowly withdrawing with a syringe, leaving the cake in the original vial. The infranatant was then placed in a fresh 100 mL glass vial and centrifuged for 10 minutes at 1000×G under a decafluorobutane gas headspace, causing substantially all of the microbubbles to migrate into the cake chamber. The infranatant was collected as above and discarded; the remaining microbubbles were re-suspended by gently agitation in saline/glycerin/propylene glycol buffer at a concentration of 2E9 per mL and stored in 3 mL vials under a headspace of 2 mL decafluorobutane gas at 4-8 deg C. The microbubble diameter was assessed by electrozone sensing periodically (Coulter Counter 4: Beckman-Coulter). The small diameter microbubbles prepared by this method were found to be stable on storage, with less than 15% change in mean diameter over 3 months.

### Example 8

### Preparation of Large Microbubbles with Symmetric Size Distribution

Microbubbles exhibiting a symmetric size distribution, with a mean diameter of 2.3 m with less than 35% below 1.8 μm and less than 5% above 4 μm, were prepared as follows. PDP-bearing microbubbles were synthesized as described in Example 2, and diluted to a concentration of 5E9 per mL in 10 mL of saline/glycerin/propylene glycol buffer. Microbubbles were centrifuged in a 50 mL glass vial containing a headspace of decafluorobutane gas for 1 minute at 500×G, and 9 mL of infranatant was collected and discarded. 9 mL of saline/glycerin/propylene glycol buffer was added to the vial and the microbubbles re-suspended by gentle agitation. The vial was centrifuged again for 1 minute at 500×G, and the infranatant collected and discarded. Microbubbles were resuspended in 2 mL of saline/glycerin/propylene glycol buffer and packaged in glass vials under a headspace of decafluorobutane.

### Example 9

### Lyophilization of Antibody-Conjugated Lipid Microbubbles

Microbubbles bearing an anti-CD4 antibody were prepared as described in Specific Examples 3. Microbubbles were washed into an aqueous solution of isotonic sucrose, and re-suspended at a concentration of 2E9 per mL. Microbubbles were aliquoted at 0.5 mL in 2 mL vials. Vials were frozen at −40 deg C for 10 minutes in an acetonitrile/dry ice bath. Vials were then placed in a lyophilization chamber maintained at −20 deg C, and lyophilized for 24 h under a vacuum of −1000 mbar. The resulting lyophilisate was a dried white cake. Vials capped under a headspace of decafluorobutane gas. Microbubbles were stored at room temperature.

The lyophilisate was reconstituted by adding 0.2 mL water and agitating the vial by hand or vortex. Electrozone sensing, flow cytometry and transillumination microscopy revealed the presence of microbubbles. The presence of the CD4 antibody on the reconstituted microbubble was verified by flow cytometry, and its activity by immunoblot with recombinant His-tagged CD4, as in Example 3.

### Example 10

### Synthesis of Uncharged Lipid Microbubbles Containing Air

Microbubbles can also be prepared with cores composed of a variety of gasses, including those with higher water solubility and other physical properties different than the fluorocarbon gasses typically used in the art. Air-encapsulated microbubbles may be prepared as in Specific Example 1 by substituting air for decafluorobutane, or by substituting air for the perflurocarbon headspace after lyophilization.

It should be noted that lyophilization provides a useful method for exchanging the content of the gaseous core. The gas comprising the initial core is removed by vacuum during the lyophilization process, and the vials subsequently sealed with a headspace comprised of a second gas. The core of microbubbles formed upon reconstitution of the lyophilisate will be comprised of the second gas.

For example, air-encapsulated microbubbles were prepared my modifying the lyophilization method described in Example 9. Decafluorobutane microbubbles were synthesized as in Example 1, and lyophilized as in Example 9. After lyophilizing for 24 hours under vacuum, the vials were sealed under atmospheric pressure such that the headspace within the vials comprised air. The lyophilized cake was then reconstituted by adding 0.5 mL of water to the vial and gently agitating. The presence of microbubbles was confirmed by transillumination microscopy (Zeiss Axiophot, 40× objective) and electrozone sensing.

### Example 11

### Assessment of Non-Specific Binding to Cells

Microbubbles were prepared as in Example 6, without the incorporation of an antibody. The spleen was collected from a freshly sacrificed mouse and splenocytes homogenized by passage through a 70 μm filter. The recovered cell mixture contained spleen cells in addition to leukocytes and erythrocytes. The cells were concentrated to 4E7 per mL in PBS containing 0.5% BSA and 2 mM EDTA (FACS buffer) and placed on ice. 1E7 cells were added to 1E8 microbubbles in a total volume of 300 μL and incubated at room temperature with end-to-end agitation for 10 minutes. The dispersion was then diluted to a final volume of 2.0 mL and incubated in a microslide chamber with a 100 μm depth (Microslide III-0.1; Ibidi) for 10 minutes at room temperature. The top and bottom surface of the chamber was examined under 400× magnification using transillumination. A ten minute incubation time was found to be sufficient time for all cells to settle to the bottom surface of the microchamber by sedimentation: any microbubbles found on the top surface of the microchamber were assumed to be bound to microbubbles. The number of cells on the top and bottom surface was counted for 20 optical fields of view. The binding efficiency was computed as the number of cells on the top of the chamber relative to all of the cells counted. This binding efficiency was used as a measurement of the non-specific binding capacity of the microbubbles prepared here.

A mean binding efficiency of 1% was found in n=3 microchambers for mouse splenocytes.

In a separate experiment, a mean binding efficiency of 0.5% was found for mouse splenocytes after lysis of erythrocytes.

In a separate experiment, a mean binding efficiency of 0.3% was found for whole anti-coagulated human blood collected from healthy volunteers.

### Example 12

### Collapse of Microbubbles Under Positive Pressure

Lipid microbubbles encapsulating a gaseous core of decafluorobutane gas were prepared as in Specific Example 1 at 2E9 per mL in saline/glycerin/propylene glycol buffer. Volume per volume dilutions of microbubbles in FACS buffer (1, 10, and 100%) were prepared. The plunger was removed from a 5.0 mL luer-lock syringe, and the needle hub sealed by means of a closed stopcock. Three-hundred microliters of the diluted microbubble dispersion was placed into the syringe, and the plunger re-inserted into the barrel. The plunger was depressed to a final volume of 1.0 mL, and released in 1 second intervals five times. This resulted in the generation of approximately 700 mbar of positive pressure.

Electrozone sensing did not detect any microbubbles in the 0.5-15 m diameter range in any of the treated samples. Light microscopy (400× magnification) similarly did not reveal the presence of any buoyant particles.

It will be clear to one skilled in the art that various forms of generating pressure, both positive and negative, can be used to collapse the microbubbles according to this invention. Exemplary methods include application of acoustic energy.

### Example 13

### Separation of Microbubbles from Cells Using a Two Chamber Apparatus

Lipid microbubbles were prepared as in Example 6, without the incorporation of an antibody. Microbubbles were mixed with an equal volume of fresh mouse splenocytes incubated for 10 minutes with end-to-end agitation at room temperature. The dispersion was then loaded into the upper chamber of the two-chamber apparatus shown in FIGS. 1-3, and the top was securely fastened. The lower chamber was filled with 3 mL of FACS buffer, and the upper chamber was placed onto the lower chamber. The apparatus was then centrifuged for 5 minutes at 500×G. After centrifugation, the upper chamber was removed from the lower chamber. Cells and microbubbles within each chamber were re-suspended by gentle agitation, and each re-suspended in a total volume of 5.0 mL of FACS buffer. The concentration of cells and microbubbles in each chamber was determined by flow cytometry. The upper chamber was found to be highly enriched in microbubbles, and possess essentially no cells. Nearly all of the cells were recovered in the lower chamber, and essentially no microbubbles were found in the lower chamber (FIG. 4).

### Example 14

### Isolation of CD8+ T-Cells from Mouse Splenocytes Using Antibody-Bound Microbubbles and a Two Chamber Apparatus

Microbubbles bearing an antibody against mouse CD8 were prepared as in Example 3.

The spleen was collected from a freshly sacrificed mouse and splenocytes homogenized by passage through a 70 μm filter. The recovered cell mixture contained spleen cells in addition to leukocytes and erythrocytes. The cells were concentrated to 6E7 per mL in FACS buffer and placed on ice. 1E7 cells were added to 1E8 CD8-conjugated microbubbles in a total volume of 300 uL and incubated at room temperature with end-to-end agitation for 10 minutes. The dispersion placed into the upper chamber of the two-chamber insert shown in FIGS. 1-2, and the apparatus was centrifuged as described in Example 13. After centrifugation, the upper chamber was exposed to a positive hydrostatic pressure to collapse the microbubbles, as in Example 12. Cells in the upper and lower chambers were stained with fluorescently labeled antibodies against CD4 and CD8, and the concentration of CD8+ T-cells in each fraction was assessed by flow cytometry (Guava; EMD Millipore).

No microbubbles were found on flow cytometry in either the upper or lower fraction. The upper fraction was found to be enriched in CD8+ cells, while the lower chamber was largely depleted of CD8+ cells (<4% CD8+).

### Example 15

### Separation of CD8+ T-Cells from Mouse Spleen Using an Antibody-Conjugated Microbubble

Microbubbles bearing an antibody against mouse CD8 were prepared as in Example 3. The spleen was collected from a freshly sacrificed mouse and splenocytes homogenized by passage through a 70 μm filter. The recovered cell mixture contained spleen cells in addition to leukocytes and erythrocytes. The cells were concentrated to 6E7 per mL in FACS buffer and placed on ice. 1E7 cells were added to 1E8 CD8-conjugated microbubbles in a total volume of 300 uL and incubated at room temperature with end-to-end agitation for 5 minutes. The dispersion was then diluted to a final volume of 2.0 mL in a polystyrene FACS tube. The FACS tube was then centrifuged at 300×G, 4 deg C, for 5 minutes.

Centrifugation caused the microbubbles and microbubble-bound cells to migrate in the direction opposite the applied centrifugal force, and they formed a “cake” at the top part of the tube. The cells that did not bind to any microbubbles migrated to the bottom of the tube, forming a pellet. The supernatant containing the cake was carefully harvested with a pipette, being careful not to disturb the pellet, and placed into a fresh 15 mL Eppendorf tube, the pelleted cells were resuspended in fresh FACS buffer. Samples from each fraction were assessed microscopically. The pellet fraction (“negative fraction”) contained only free cells and no microbubbles. The cake fraction (“positive fraction”) contained both free microbubbles and microbubble-bound cells, but no free cells.

The cake fraction was exposed to a pressure of 700 mbar by inserting a 10 mL syringe plunger into the Eppendorf tube. This caused the microbubbles to collapse, leaving free cells. The resulting suspension was centrifuged once at 300×G for 5 minutes, resulting in the formation of a pellet. No cake was visible. The supernatant was decanted and discarded, and the pellet was resuspended in fresh FACS buffer.

Cells from the positive, negative, and unsorted fractions were stained with fluorescently labeled antibodies against CD4 and CD8, as well as 7AAD as a viability stain and assessed by flow cytometry. Results from n=3 replicates are shown in Table 3. Purity was computed as the percent of CD8+ cells relative to CD8− cells in the positive fraction. Depletion was computed as the percent of CD8+ cells left in the negative fraction. Viability was defined as the percent of 7AAD− cells in the positive fraction. The splenocyte samples before the procedure had an average CD8+ concentration of 11%; this was enriched to an average of 84% after the separation procedure. The viability of the positively selected cells was >90% for all samples.

TABLE 7
separation of mouse CD8+ cells

| | Mean +/− StDev |
|---|---|
| Unsorted CD8% | 11 +/− 1.5% |
| Purity (%) | 92% |
| Depletion (%) | 0.7 +/− 0.3% |
| Viability (Vi-Cell) | 93 +/− 1.9% |

This experiment was repeated using a two-chamber apparatus, and the positive fraction collected by decantation followed by microbubble collapse. The duration of the procedure was reduced by approximately 50%, as the tedious manual harvesting of the buoyant cake was not necessary. A purity of >90% and yield of ˜50% was achieved.

### Example 16

### Separation of CD8+ T-Cells from Human Blood Using an Antibody-Conjugated Microbubble

Microbubbles bearing an antibody against human CD8 were prepared as in Example 3. Blood was collected from healthy volunteers in a EDTA vacutainer. Erythrocytes were lysed using a commercially available lysis kit (RBC Lysis Buffer, eBioscience) and the remaining cells were resuspended at 4E7 per mL in FACS buffer. 1E7 cells were added to 5E7 CD8-conjugated microbubbles in a total volume of 300 uL and incubated at room temperature with end-to-end agitation for 10 minutes. The dispersion was then diluted to a final volume of 2.0 mL and then centrifuged at 300×G, 4 deg C, for 5 minutes. Positive and negative fractions were collected, and cells were stained for CD8, CD4, and 7AAD as a viability marker. This experiment was repeated with blood from n=3 donors.

Flow cytometry revealed that samples before the procedure had an average CD8+ concentration of 23%; this was enriched to an average of 94% after the separation procedure. The viability of the positively selected cells was >90% for all samples. Positively selected cells and untouched cells were preserved for 12 hours at 4 deg C. The viability of the positively selected cells was not significantly different than that of the untouched cell population at this time point.

TABLE 8
separation of human CD8+ cells.

| | Mean +/− StDev |
|---|---|
| Unsorted CD8% | 23 +/− 13.3% |
| Purity (%) | 94 +/− 3.8% |
| Depletion (%) | N/A |
| Viability (Vi-Cell) | 99 +/− 0.6% |

### Example 17

### Separation of CD19+ B-Cells from Mouse Spleen Using an Antibody-Conjugated Microbubble

Microbubbles bearing an antibody against mouse CD19 were prepared as in Example 3. The spleen was collected from a freshly sacrificed mouse and splenocytes homogenized by passage through a 70 μm filter. Erythrocytes were lysed using a commercially available lysis kit (RBC Lysis Buffer, eBioscience) and the remaining cells were resuspended at 4E7 per mL in FACS buffer. 1E7 cells were added to 5E7 CD19-conjugated microbubbles in a total volume of 300 uL and incubated at room temperature with end-to-end agitation for 10 minutes. The dispersion was then diluted to a final volume of 2.0 mL and the centrifuged at 200×G, 4 deg C, for 2 minutes followed by 500×G for 2 minutes. Positive and negative fractions were collected. Cells were stained for CD8, CD4, and 7AAD as a viability marker.

Flow cytometry revealed that samples before the procedure had an average CD19+ concentration of ˜60%; this was enriched to >95% after the separation procedure. The viability of the positively selected cells was >90% for all samples

### Example 18

### Separation of CD4+ T-Cells Using a Second Antibody and “Universal” Streptavidin Microbubble

T-cells were isolated from a complex mixture derived from spleen homogenate as follows. The spleen was collected from a freshly sacrificed mouse and splenocytes homogenized by passage through a 70 μm filter. The recovered cell mixture contained spleen cells in addition to leukocytes and erythrocytes. The cells were concentrated to 6E7 per mL and placed on ice. A biotinylated anti-mouse CD4 antibody (clone GK1.5, eBiosciences) was added to the cells at 1 μg per 10E7 cells, followed by incubation on ice for 20 minutes. Cells were then washed once to remove free antibody. Cells were then added to streptavidin-coated microbubbles prepared as in Example 4 at a ratio of 5 microbubbles per cell. The cell-microbubble dispersion was incubated with the microbubbles for 20 minutes at 4 degree C with gentle agitation. The cell-microbubble dispersion was transferred to a polystyrene FACS tube and diluted to 1 mL with FACS buffer (2.0 mM fetal bovine serum in DPBS), then centrifuged for 5 minutes at 300×G. This resulted in the formation of a cake composed of cell-microbubble complexes at the top of the tube, and a pellet composed of cells at the bottom of the tube. The cake was carefully decanted, and the cell-microbubble complexes re-dispersed in FACS buffer.

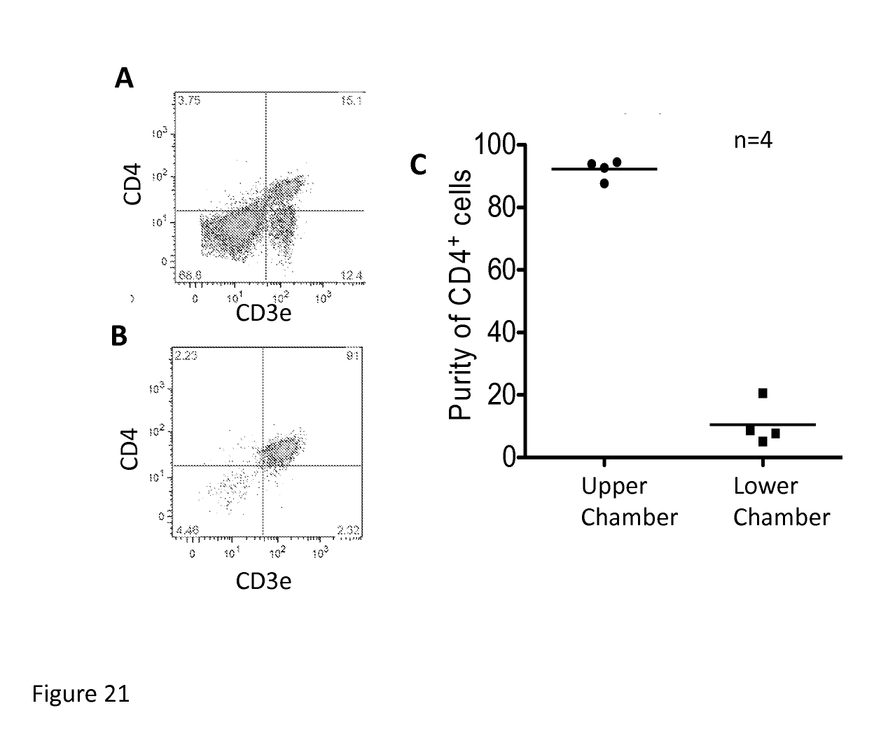

The cells were stained for CD4 (clone RM4-4), CD3e (clone 145-2C11), and CD45 (clone 30-F11) and analyzed by flow cytometry. The cells attached to the microbubbles were found to be CD4+ T-cells with a purity of 95% (n=4). These results are demonstrated in FIG. 21.

It should be noted that this procedure may be used for virtually any cell surface molecules for which an antibody or other type of targeting ligand is available. Other cell surface molecules of interest include but are not limited to cluster of differentiation designated molecules such as CD1, CD2, CD3, CD4, CD5, CD6, CD8, CD10, CD11b, CD14, CD16, CD19, CD22, CD23, CD24, CD25, CD27, CD28, CD30, CD31, CD33, CD34, CD38, CD41, CD43, CD45, CD45R, CD49, CD56, CD61, CD62L, CD66, CD69, CD71, CD90.1, CD90.2, CD105, CD117, CD127, CD133, CD134, CD137, CD138, CD146, CD154, CD162, CD184, CD294, CD326, surface markers of apoptosis such as phosphatidylserine, chemokine receptors, cell surface glycoproteins, and cell adhesion molecules.

### Example 19

### Assessment of Viability of Isolated Cells

CD8+ T-cells were isolated from mouse splenocytes as in example 15. After isolation, the cells were diluted in FACS buffer and stored on ice for 24 hours. Viability was assessed immediately after isolation and at 24 hours by trypan blue exclusion with an hemacytometer, by automated trypan blue cell counting (Vi-Cell, Beckman-Coulter), and by flow cytometry (Vi-Count; EMD Millipore). No statistically significant difference was found between the unsorted and positively selected cells at either time point by any of the viability methods assessed (n=3 replicates).

### Example 20

### Low Melting Point Microbubbles

Detachment of microbubbles from positively-selected cells may be achieved by collapsing the cell-microbubble complexes at a temperature greater than the main phase transition temperature (melting point) the microbubble lipid shell. Low melting point microbubbles can be synthesized by using a low transition temperature lipid, such as dipalmitoylphosphatidylcholine (˜33 degrees C), as the primary shell component. 37.1 mg of dipalmitoylphosphatidylcholine, 20 mg polyoxyethylene 40 stearate, and 5 mg of PDP-PEG(2000)-disteroylphosphatidylethanolamine (Avanti) is added to 20 mL of sterile normal saline (Baxter) and sonicated to clarity using a probe-type sonicator. Microbubbles are synthesized as in Example 1, and antibody is conjugated to the microbubbles as in Example 3. Antibody-labeled cells are used to isolate cells as in Examples 13-17. The cell-microbubble complexes are decanted into a 1.5 mL Eppendorf tube and placed in a 37 degree cell incubator for 2-60 minutes. Exposure to this temperature causes the main lipid of the shell to exist predominantly in the liquid expanded phase. The microbubbles are then collapses by positive pressure, and residual shell components removed from the suspension by washing 5 minutes at 500×G in fresh FACS buffer.

### Example 22

### Use of Acoustic Radiation Force to Separate Microbubble-Cell Complexes

Acoustic radiation force can be used in lieu of buoyancy to affect a very rapid separation of Cell-Microbubble complexes from free cells. This mechanism takes advantage of the ability of acoustic radiation force (also known as Bjerkness forces) to exert a translational displacement of gas-encapsulated microbubbles. Microbubbles bearing an antibody are prepared as in Example 3, and incubated with the heterogeneous cell population as in Examples 13-17. The dispersion is then diluted to 10 mL in a beaker from which the bottom has been replaced with mylar or another acoustically permeable material. The beaker is placed into a holder that sits above an ultrasound transducer operating at approximately 0.1-10 MHz, an acoustic pressure of approximately 50-500 kPa. The ultrasound transducer is turned on for 1-100 seconds, thereby causing the microbubble-cell complexes to translate away from the transducer toward the top of the beaker, forming a cake. The cake is then harvested, and cells may then be used with or without removal of microbubbles as described in Example 13-18.

### Example 23

### Isolation of Soluble Analytes

Antibody-bearing microbubbles prepared as in Example 3 can be used to isolate or concentrate soluble targets using the methods described in Examples 13-18 and 22. For example, soluble antibody produced by hybridoma cells may be isolated as follows. Microbubbles prepared as in Example 3 with an antibody ligand reactive for rat IgG1 are incubated with hybridoma cells that secrete a monoclonal antibody derived in rat and of isotype IgG1, or in the supernatant after removal of the cells. The dispersion is then centrifuged and microbubbles collected as described in Examples 13-18.

It will be clear to one skilled in the art that numerous other soluble components can be isolated using this method, providing that a targeting ligand specific for said analyte exists. Exemplary soluble analytes are nucleic acids, lipids, sugars, hormones, antibodies, cytokines, and other substances secreted by cells.

### Example 24

### Negative Selection for Isolation of Desired Cells

Desired cells can be isolated from a complex mixture by negative selection in the context of the present invention. This can be achieved using a two-step separation scheme, in which soluble ligands, such as antibodies, specific for the un-desired cell(s) are first added to the mixed cell population, and the antibody-labeled cells are subsequently isolated and discarded using a “universal” microbubble. The antibodies must bear a suitable marker group, thereby enabling selection using a microbubble bearing the appropriate ligand. The following example illustrates this process for isolation of CD4+ cells from murine spleen.

Fresh spleen homogenate is incubated for 5 minutes with a cocktail of phycoerythrin (PE)-labeled antibodies which collectively label all non-CD4+ cell type found within the spleen: CD8a, CD11b, CD11c, CD19, B220, TCR g/d, and TER119. Un-bound antibody is then removed by washing the cells in FACS buffer, and cells are re-suspended at 4E7 per mL in FACS buffer. Cells are incubated with microbubbles bearing an anti-PE antibody at a ratio of 5:1 for 10 minutes with gentle agitation. The dispersion is then placed into the two-chamber insert and centrifuged for 5 minutes. The upper chamber, containing the microbubble-bound cells (targeted fraction) is discarded and the lower chamber, containing the desired CD4+ cells, is retained.

It should be noted that negative selection can also be performed in a single step by utilizing a panel of microbubbles bearing antibodies against the cell types desired to be removed.

### Example 25

### Sequential Selection Using More than One Microbubble Formulation

Separation procedures occurring in sequence are also contemplated in this invention. The collapsible microbubble in conjunction with the two-chamber device makes sequential separation feasible, in that desired cells may be positively selected with a first microbubble, said microbubbles then collapsed and the cells collected, and the collected cells then selected using a second microbubble with specificity for a second target. This may be particularly useful for isolating complex cells defined by the expression of more than one cell surface marker, and found in a mixed cell population in which unwanted cells express one or more of said cell surface markers.

For example, regulatory T-cells are defined by the expression of both CD4 and CD25 (CD4+/CD25+). Each of these cell surface markers (CD25 and CD4) are expressed separately on different cell types found in mouse spleen homogenate. Thus, isolation using a microbubble with specificity to a single target (CD4 or CD25), or using both microbubble formulations at the same time (CD4 and CD25) will result in contamination of the desired double positive (CD4+/CD25+) cells with unwanted single positive cells (CD4+/CD25− and or CD4−/CD25+). The desired CD4+/CD25+ cells can be isolated as follows. The splenocytes are incubated with a first microbubble formulation comprising a microbubble bearing an anti-CD4 antibody, and a positive selection is performed as described in Specific Example 14. The targeted cells collected in the upper chamber will consist of the desired CD4+/CD25+ cells, in addition to unwanted CD4+/CD25− cells. The cell-bound microbubbles are collapsed as described in Specific Example 12, The collected cells are then incubated with a second microbubble formulation, comprising a microbubble bearing an anti-CD25 antibody, and positive selection performed as in Specific Example 14, and the microbubbles collapsed. The targeted cells collected in the upper chamber now consist of the desired CD4+/CD25+ cells, while all CD25− cells will be found in the lower chamber.

The aforementioned sequential separation procedure may be performed with any number of steps, and in various combinations of positive and negative selection. For example, CD127−/CD4+/CD25+ T-cells may be isolated from mouse splenocytes by first performing a positive selection with a microbubble bearing an anti-CD127 antibody, and discarding the positive fraction containing all CD127+ cells. The negative fraction of cells (in the lower chamber) then undergo two sequential rounds of positive selection with microbubbles bearing an anti-CD4 and anti-CD25 antibody, as described above.

It should be clear that the aforementioned sequential separation procedure may be performed using soluble antibodies bearing distinct marker groups and “universal” microbubbles. For example, CD4+/CD25+ cells may be isolated by first incubating splenocytes with a biotin-anti-CD4 antibody and a PE-anti-CD25 antibody. The cells are then washed to remove free antibody, and a positive selection performed as described above with a microbubble bearing an anti-biotin antibody. The targeted cells retained in the upper chamber are collected and microbubbles collapsed, and a second positive selection is performed on the cells with a microbubble bearing an anti-PE antibody.

It should be noted that non-buoyancy based separation methods may be used in conjunction with the sequential separation procedure described in this example. For example, in some cases it may be efficacious to first perform a negative selection for erythrocytes using TER119-labelled magnetic particles, and second perform a positive selection for CD4+ cells.

### Example 26

### Separation in a Sterile and Closed System

Separation procedures occurring in a sterile and closed system, for example in the context of isolating cells for use as a therapeutic, are contemplated. The microbubbles and two-chamber device of the instant invention are used to isolate CD34+ stem cells from human cord blood as follows. Anti-coagulated and erythrocyte-lysed cord blood is drawn into a sterile syringe, and added to a sterile two-chamber apparatus comprising a leur-lock port. Sterilized microbubbles comprising a targeting ligand specific for CD34 are then added to the same chamber via a sterile syringe and luer-lock port. Sterile PBS is added to the opposite chamber via a luer-lock port. Cells and microbubbles are incubated in the apparatus, the apparatus is centrifuged to effect positive selection of the CD34 cells. The microbubbles are collapsed in the insert by connecting a sterile syringe and applying a positive hydrostatic pressure. The buoyant fraction in the top chamber is re-suspended by gentle agitation, and collected through a luer-lock port with a fresh syringe. The CD34+ cells are then administered to the patient.

### Example 27

### Determination of Number of Microbubbles Required to Float Cells

Microbubbles were synthesized with a 1% anchor group and conjugated to an anti-mouse CD19 antibody (<50,000 antibodies per MB). The mean diameter microbubbles in the dispersion was approximately 3 um. Fresh splenocytes were incubated with a ratio of 10 MB per cell for 5 minutes at room temperature with rotational and end-to-end mixing. The dispersion was then loaded into an IBIDI microslide, having a depth of 100 um. The microbubbles and cells were allowed to settle for 5 minutes, then the top focal plane and bottom focal plane were visualized by transillumination microscopy at with 20× long working distance objective. Both free microbubbles and microbubbles bound to cells were observed on the top plane, demonstrating that cells on the top plane migrated with free microbubbles and were therefore buoyant. The number of microbubbles per cell on the top plane was computed. No free microbubbles were observed on the bottom focal plane, although occasional cells with one or more attached microbubble were found. The average number of microbubbles per cell was measured for sedimented cells with an attached MB. It was found that, on average, 9 MB were attached to every buoyant cell. For sedimented cells that had any MB bound, there were between 1 and 3 microbubbles per cell.

### REFERENCES

Moore, A., R. Weissleder, and A. Bogdanov, Uptake of dextran-coated monocrystalline iron oxides in tumor cells and macrophages. Journal of Magnetic Resonance Imaging, 1997. 7(6): p. 1140-1145.
Faraji, A. H. and P. Wipf, Nanoparticles in cellular drug delivery. Bioorganic & Medicinal Chemistry, 2009. 17(8): p. 2950-2962.
Pisanic, T., et al., Nanotoxicity of iron oxide nanoparticle internalization in growing neurons. Biomaterials, 2007. 28(16): p. 2572-2581.
Berry, C. C., et al., Dextran and albumin derivatised iron oxide nanoparticles: influence on fibroblasts in vitro. Biomaterials, 2003. 24(25): p. 4551-4557.
Rychak J J, Lindner J R, Ley K, Klibanov A L. Deformable gas-filled microbubbles targeted to P-selectin. J Control Release. 2006 Sep. 12; 114(3):288-99.
Simberg D, Mattrey R. Targeting ofperfluorocarbon microbubbles to selective populations of circulating blood cells. J Drug Target. 2009 June; 17(5):392-8.
Shi G, Cui W, Benchimol M, Liu Y T, Mattrey R F, Mukthavaram R, Kesari S, Esener S C, Simberg D. Isolation of rare tumor cells from blood cells with buoyant immuno-microbubbles. PLoS One. 2013; 8(3):e58017.
Shi G, Cui W, Mukthavaram R, Liu Y T, Simberg D. Binding and isolation of tumor cells in biological media with perfluorocarbon microbubbles. Methods. 2013 Dec. 1; 64(2): 102-7.
Hus C H, Chen C C, Irimia D, Toner M. Isolating cells from blood using buoyancy activated cell sorting (BACS) with glass microbubbles. 14^{th} International Conference on Miniaturized Systems for Chemistry and Life Sciences. 3-7 Oct. 2010, Groningen, The Netherlands. Pp 226-228.
Rychak J J, Lindner J R, Ley K, Klibanov A L. Deformable gas-filled microbubbles targeted to P-selectin. J Control Release. 2006 Sep. 12; 114(3):288-99.
Klibanov et al., U.S. Patent Application No. 2005/0260189 A1.
Wilson et al., U.S. Patent Application No. 2012/0288852 A1.
Rongved et al., U.S. Patent Application No. 2007/0036722 A1.
Cuthbertson et al., U.S. Patent Application 2003/0104359 A1.
Hossack et al., U.S. Patent Application No. 2014/0142468 A1.
Rychak. U.S. Patent Application No. 2012/0244078 A1.
Klaveness et al., U.S. Pat. No. 6,261,537.
Klibanov et al., U.S. Pat. No. 6,245,318.
Unger et al., U.S. Pat. No. 6,139,819.
Mattrey et al., International (PCT) Publication No. WO2009052057 A1.
Toma et al., U.S. Pat. No. 8,460,269.
Kulseth et al., U.S. Pat. No. 6,806,045.
Jablonski et al., U.S. Pat. No. 8,835,186.
Jablonski et al., U.S. Pat. No. 8,513,032.
Delaage et al., U.S. Pat. No. 5,116,724.

What is claimed is:

1. A method of separating target cells from a mixed cell population in a liquid sample using a two chamber apparatus, the method comprising the steps of:
i. mixing the cells with a buoyant microbubble composition in the liquid sample;
ii. incubating the liquid sample at a temperature between 4° C. and 37° C. for a sufficient time to allow the target cells and the microbubbles to form cell-microbubble complexes;
iii. adding the liquid sample to the two-chamber apparatus;
iv. applying sufficient centrifugal force to the liquid sample containing the cell-microbubble complexes in said two-chamber apparatus for a sufficient period of time to cause the cell-microbubble complexes to become enriched in the upper chamber of said two-chamber apparatus, and the remaining cell population to become enriched in the bottom chamber of said two-chamber apparatus;
v. exerting sufficient pressure to said top chamber to collapse the microbubbles, thereby liberating the target cells from the microbubble-cell complex; and
vi. collecting the target cells.

2. The method of claim 1 whereby the free cells are collected.

3. The method of claim 1 wherein the time period for incubating the liquid sample is between 1 and 60 minutes.

4. The method of claim 1 wherein the time period for applying centrifugal force to the liquid sample is between 0.1 and 60 minutes.

5. The method of claim 1 wherein the pressure is in the form of hydrostatic pressure and is applied by decreasing the volume of the top chamber of the apparatus by depressing a plunger.

6. The method of claim 1 wherein the relative centrifugal force is between 1 and 500.

7. A method of separating target cells from a mixed cell population in a liquid sample using a two chamber apparatus, the method comprising the steps of:
i. mixing the cells with an aqueous solution containing more than one ligand, each labeled with a distinct marker group, to form a suspension;
ii. mixing the suspension from step (i) with a first buoyant microbubble composition, wherein said first microbubble composition comprises a ligand specific for one of the marker groups;
iii. incubating the liquid sample at a temperature between 4° C. and 37° C. for a sufficient time to allow the target cells and the microbubbles to form cell-microbubble complexes;
iv. adding the liquid sample to the two-chamber apparatus;
v. applying sufficient centrifugal force to the liquid sample containing the cell-microbubble complexes in said two-chamber apparatus for a sufficient period of time to cause the cell-microbubble complex to become enriched in the upper chamber of said two-chamber apparatus, and the remaining cell population to become enriched in the bottom chamber of said two-chamber apparatus;
vi. exerting sufficient pressure to said top chamber to collapse the microbubble, thereby liberating the target cells from the microbubble-cell complexes;
vii. collecting the target cells.
viii. mixing the collected target cells with a second buoyant microbubble composition, wherein said second microbubble composition comprises a ligand specific for a different marker group;
ix. repeating steps ii-viii one or more times until the desired target cells bearing all marker groups have been collected.

8. The method of claim 7 whereby step ix is repeated between 1 and 3 times.

9. The method of claim 7 whereby the cells in the bottom chamber are collected.

10. A method of separating a soluble analyte from an aqueous sample using a two chamber apparatus, the method comprising the steps of:
i. mixing the aqueous sample with a buoyant microbubble composition in a liquid sample,
ii. incubating the liquid sample at a temperature between 4° C. and 37° C. for a sufficient time to allow the soluble analyte and the microbubbles to form analyte-microbubble complexes;
iii. adding the liquid sample to the two chamber apparatus;
iv. applying sufficient centrifugal force to the liquid sample containing the analyte-microbubble complexes in said two-chamber apparatus for a sufficient period of time to cause the cell-microbubble complexes to become enriched in the upper chamber of said two-chamber apparatus, and the remaining non-buoyant material to become enriched in the bottom chamber of said two-chamber apparatus; and
v. collecting the contents of the upper chamber and/or the bottom chamber.

11. A two-chamber apparatus for use in separating target cells comprising a first top chamber with a cylindrical shape and an opening at one end and further comprising a means for sealing said opening, a second bottom chamber with a cylindrical shape and further comprising a rounded or conical closed end, and wherein a tapered insert separates said top chamber from said bottom chamber, and wherein said top chamber can be detached from said bottom chamber.

12. The two-chamber apparatus of claim 11 wherein the bottom chamber of said apparatus comprises a conical centrifuge tube.

13. The two-chamber apparatus of claim 11 wherein said means for sealing the open end of the top chamber comprises one or more normally closed valve wherein the closed valve is opened as desired by a user.

14. A gas-encapsulated microbubble composition for use in isolation of biological substances outside of the body, comprising a lipid monolayer shell and a targeting ligand, wherein said targeting ligand density is between 1 and 50,000 molecules per microbubble.

15. The composition of claim 14, wherein the microbubble shell comprises two shell-forming surfactants, a first surfactant and a second surfactant having a higher water solubility than said first surfactant and wherein said first surfactant is present in the shell in a moles/moles ratio of 50-75% relative to other shell components, wherein said second surfactant is present in the shell in a moles/moles ratio of 15-50%, relative to other shell components.

16. The composition of claim 14 wherein said gas core is selected from group consisting of air, nitrogen, argon, sulfur hexafluoride, perfluoroethane, perfluoropropanes, perfluorobutanes, perfluorocyclobutanes, perfluoropentanes, perfluorocyclopentanes, perfluoro methylcyclobutanes, perfluorohexanes, perfluorocyclohexanes, perfluoro methyl cyclopentanes, perfluoro dimethyl cyclopentanes, perfluoro heptanes, perfluoro cycloheptanes, perfluoro cycloheptanes, perfluoromethyl cyclohexanes, perfluoro dimethyl cyclopentanes, perfluoro trimethyl cyclobutanes, perfluoro triethylaminesperfluoropropane, perfluorobutane and similar, or a mixture thereof.

17. The composition of claim 14 wherein the targeting ligand further comprises an anchor molecule selected from the group consisting of lipids, phospholipids, long-chain aliphatic hydrocarbons, lipid multichains, comb-shaped lipid polymer steroids, fullerenes, polyaminoacids, native or denatured proteins, aromatic hydrocarbons, fatty acids, or partially or completely fluorinated lipids, and PEG-derivatized versions of the above.

18. The composition of claim 14 wherein the microbubble shell undergoes a phase transition at between 30-38 degrees C.

19. The composition of claim 14 wherein the microbubble shell undergoes a phase transition at between 15-38 degrees C.

20. The composition of claim 14 wherein the microbubble shell comprises between 50 and 90% by moles a lipid having a main phase transition temperature of between 0 and 38 degrees C, and wherein the remaining shell components have a main phase transition temperature of greater than 38 degrees C.

21. The composition of claim 14 wherein the microbubble shell comprises between 1 and 40% by moles a lipid having a main phase transition temperature of between 0 and 38 degrees C, and wherein the remaining shell components have a main phase transition temperature of greater than 38 degrees C.

22. The composition of claim 14 wherein the microbubble shell comprises between 1 and 15% of a PEG-grafted lipid.

23. The composition of claim 15 wherein the second surfactant is selected from the group consisting of: fatty acids and salts thereof, sugar esters of fatty acids, PEG-phospholipids, PEG-stearate, DSPE-PEG-2000, DSPE-PEG-350, or DSPE-PEG-1000.

24. The composition of claim 14 wherein the targeting ligand is a hormone, amino acid, peptide, peptidomimetic, protein, nucleic acid, deoxyribonucleic acid, ribonucleic acid, lipid, antibody or antibody fragment, carbohydrate, aptamer, or combination thereof.

25. The composition of claim 14 wherein the microbubble shell comprises essentially no surface charge.

26. The composition of claim 14 wherein the average microbubble diameter is between 3 and 5 um.

27. The composition of claim 14 wherein the average microbubble diameter is between 1 and 2 um.
